(19) United States
(12) Patent Application Publication
(10) Pub. No.: US 2025/0302403 A1
(43) Pub. Date: Oct. 2, 2025

(54) REMOTE VIRTUAL VISITATION AND INFORMATION EXCHANGE

(71) Applicant: VOICELOVE, LLC, Scarsdale, NY (US)

(72) Inventors: Gabriela PELIN, Winter Park, FL (US); Sarah ATERETH, New York, NY (US); Gary WORD, Franklin, TN (US); Tamatha FENSTER, Scarsdale, NY (US); Marc SCHIFFMAN, New York, NY (US); Stanislav SAMARIN, Roswell, GA (US)

(21) Appl. No.: 19/094,590

(22) Filed: Mar. 28, 2025

Related U.S. Application Data
(60) Provisional application No. 63/571,310, filed on Mar. 28, 2024.
(60) Provisional application No. 63/750,714, filed on Jan. 28, 2025.

(51) Int. Cl. A61B 5/00; G06F 3/01; G06F 40/51; G06F 40/58; G06V 40/20; G16H 40/67; G16H 80/00; H04L 51/063

(52) U.S. Cl. CPC A61B 5/7475; A61B 5/7435; A61B 5/746; G06F 3/013; G06F 40/51; G06F 40/58; G06V 40/20; G16H 40/67; G16H 80/00; H04L 51/063

(57) ABSTRACT

The present invention relates to a communication system and method designed for patients in clinical settings who face challenges using standard communication devices. This system includes a mobile application and associated devices that establish a secure, asynchronous communication channel between clinicians, patients, and their support networks. Authorized users can record and send voice messages and other media, which are automatically played on the patient's device without requiring active operation. The system is tailored for ease of use, accommodating patients with severe physical or cognitive limitations through hands-free operation and simple commands. It adapts to the patient's condition, pausing messages during rest or medical procedures and resuming when appropriate. The system also features cross-linguistic messaging, translating and synthesizing voice messages across languages while maintaining the speaker's voice. Additional features include support for rich media, automatic transcription, and AI-driven content analysis. Secure, encrypted channels ensure privacy, complying with healthcare regulations. This invention enhances emotional connection and support for isolated patients, acting as a virtual visitation tool.

### CROSS-REFERENCE TO RELATED APPLICATIONS

This application claims priority under 35 U.S.C. § 119 (e) to U.S. Provisional Patent Application Nos. 63/571,310, filed Mar. 28, 2024 and 63/750,714, filed Jan. 28, 2025. The entireties of the provisional applications are incorporated by reference herein.

### GOVERNMENT LICENSE RIGHTS

This invention was made with government support under Award No. 1R42AG080891-01 awarded by the Department of Health and Human Services. The government has certain rights in the invention.

### FIELD OF THE INVENTION

The present inventions pertain to the field of communication technologies, with a specific emphasis on innovative systems, processes, methods, and apparatus designed to facilitate meaningful connections between individuals in diverse circumstances where traditional communication may be limited or challenging. This includes, but is not limited to, virtual visitation platforms, asynchronous communication modalities, and novel methods utilizing voice recordings for conveying messages of love, support, and information.

The inventions disclosed herein prioritize minimizing the effort, technical ability, physical ability, or cognitive capacity required by the receiver (and/or the sender) to engage with and benefit from the communication. This focus enables communication with individuals who may have difficulty operating traditional devices due to physical or cognitive limitations, age, or other factors. The technologies encompassed by this invention find applications in various settings, including but not limited to facilitating communication with patients in healthcare environments, children, infants, non-verbal individuals, and even animals.

Furthermore, the inventions extend to advanced features such as message queuing and storage, automated message tagging using artificial intelligence (AI), sentiment analysis, language translation, and the integration of music or media libraries. The potential integration with virtual reality (VR) and AI environments further enhances the scope and versatility of the communication experience.

One exemplary use for aspects of the invention is to communicate with someone that may have difficulty in working the controls to receive a message, such as a patient that is delirious, has dementia, or is unable to interact with traditional devices due to physical limitations or other issues. The invention supports the queuing and storing of messages for later playback. Additional features include automatic tagging of a message using AI on a transcript (including keyword and sentiment tags) and translation from the original language to an alternate language, followed by voicing the translated message using the voice of the original speaker. Messages may include songs or music from a music streaming service, AI-generated music, or music otherwise sourced. Integration with a VR environment or AI environment, in some versions of the system, is also disclosed.

### BACKGROUND

Families cannot easily or frequently visit patients in intensive care units (ICUs). This is especially true if the patient has an infectious disease (such as COVID-19, tuberculosis, influenza, measles, or C. diff) or has a compromised immune system. Patients in ICUs can become lonely. When the patient is an infant or a child, they may feel isolated and their parents can suffer from separation anxiety and parental role alteration. Family members similarly suffer, and the patient may experience infrequent or absent communication. It is understood that isolation is a significant contributor to delirium in ICU patients.

Regular cell phones are not allowed in most ICUs due to contamination and Health Insurance Portability and Accountability Act (HIPAA) concerns, and to a lesser extent due to electromagnetic interference with hospital equipment. Additionally, many patients cannot use a regular cell phone even if they have one. This may be because they are critically ill, disoriented, too young, non-verbal, deaf or hearing impaired, technophobic, visually impaired, cognitively impaired, or suffering a mental illness that interferes with device use. They may have poor or no cellular coverage, tremors, be incarcerated, or be in institutions where cell phone use is restricted or otherwise impractical.

The costs of the existing state of communication technology are manifest. According to multiple studies with years of follow-up, maintaining social connections increases the odds of survival by 50%. There is a clear need for an innovative technology to connect a patient directly with their family and friends, optionally with no intermediaries, which also reduces the burden on clinicians. Such a solution should allow private communications, help with mother-infant bonding at a much lower cost compared with routine in-person visits, and save time for clinical staff. This approach would also improve health outcomes by boosting survival through increased social capital, reduce postpartum depression, improve pediatric development, lower delirium duration and risk, and reduce patient loneliness and isolation. There is a strong need for a novel approach to facilitate communications and interactions that ease these human (and financial) costs.

### SUMMARY OF THE INVENTION

The present invention provides a communication system and method that enable patients-particularly those in clinical or care settings who cannot easily use standard communication devices-to remain closely connected with family and caregivers. In general, the system includes a mobile application (or “app”) and associated devices that create a secure, asynchronous communication channel between clinicians, patients, and their family or support network. Voice messages and other media can be recorded by authorized users and delivered automatically to a patient's device, where they are played back for the patient without the patient needing to actively operate the device. The communications channel is secure and employs robust encryption to protect privacy.

Ease of use for patients: The system is designed to be usable even by individuals with severe physical or cognitive limitations. For example, the patient's device may be configured for hands-free operation, responding to simple voice commands or minimal movements if the patient cannot press buttons. Clinicians can send updates to family members with a quick voice message instead of having to place phone calls, and patients' loved ones can easily send encouraging voice or video messages that play for the patient. The app and device together act as a “virtual visitation” tool, akin to a hands-free walkie-talkie that connects ICU patients (or others in isolation) with family and friends.

Adaptive and flexible communication: The system intelligently adapts to the patient's condition and environment. It can automatically pause or delay message playback when the patient is resting, undergoing a medical procedure, or otherwise should not be disturbed. For instance, if a patient is asleep or sedated (as indicated by connected monitors or sensors), incoming messages can be queued for later playback when the patient awakens. Conversely, when the patient is alert, queued messages will resume playing. The system can also detect situations such as a caregiver conversation or lights out in the patient's room, and correspondingly suppress or postpone non-urgent messages to avoid interference. In emergency situations, the patient can trigger an alert: for example, by speaking a help keyword that notifies clinical staff and, if not canceled, alerts family members as well.

Cross-linguistic messaging: A significant aspect of the invention is its ability to bridge language barriers automatically. Voice messages can be transcribed to text and translated from one language to another, then re-synthesized as speech in the voice of the original speaker or another selected voice. This allows, for example, a clinician to record a message in English and have the system deliver it in Spanish (using a synthesized voice that still sounds like the clinician) to a patient's family member who prefers Spanish. If no translation is needed, the original audio plays immediately; otherwise, the system handles the translation and voice conversion before playback. Similarly, patients can speak in their native language and family members afar can hear the message translated into their own language. Important terms (such as medical terminology) in messages can also be explained or translated on request, aiding understanding across language differences.

Additional features and benefits: The communication channel supports rich media beyond just voice. Messages may include video clips, music or calming sounds, live photos or other sensory data (such as a gentle vibration or lights for notification), or even VR scenes in some implementations. All messages are automatically transcribed, allowing them to be indexed, searched, and filtered by keywords or tags. Users (either senders or receivers) can tag messages with categories (for example, “love,” “urgent,” or custom labels) to organize and filter content. An AI module can analyze message content and tone, automatically tagging messages with sentiments or keywords (e.g., detecting if a message is happy, or if the patient's response indicates discomfort). The system can also integrate data from health devices or wearables to enhance communication: for instance, a wearable sensor could signal the system to adjust volume or content if the patient's heart rate or stress level is elevated. All communication is done over secure, encrypted channels to protect privacy, and the system is designed to comply with healthcare privacy regulations. By combining these features, the invention provides a lifeline for comfort and emotional connection, extending the reach of loved ones and caregivers to patients who would otherwise be isolated.

### BRIEF DESCRIPTION OF THE DRAWINGS

FIG. 1 shows an example of the primary message processing components and flow.

FIG. 2 shows the data module components and an example communication flow between a clinician and a patient's proxy, including optional language translation.

FIG. 3 shows the AI subsystem components and flow.

FIG. 4 shows an example system architecture with the main active components of the invention.

### ABBREVIATIONS AND DEFINITIONS

To facilitate understanding of the invention, some terms as used herein are defined as follows:

Clinician: A staff member or caregiver that provides care for the patient. In this context, a clinician is typically a healthcare provider (e.g. nurse or doctor) who can use the system to send or receive updates regarding the patient.

Communication Channel: A private communication link that connects one or more users with the patient. A channel typically includes the patient (on their device) and may include the patient's proxy and various family members or friends. Each channel represents a group of participants sharing messages related to one patient.

Family: As used herein, “family” refers broadly to any persons in the support network of a patient or user of the system. This can include relatives as well as close friends, clergy, caregivers, or others who the patient considers part of their support community.

HIPAA: Refers to the U.S. Health Insurance Portability and Accountability Act. Unless the context clearly requires otherwise, references to HIPAA herein are intended to encompass analogous medical privacy laws in other jurisdictions as well (for example, Ontario's Personal Health Information Protection Act, New Zealand's Health Information Privacy Code, California's Confidentiality of Medical Information Act, the Texas Medical Records Privacy Act, etc.).

Invention: When the term “invention” is used in the singular form, it should be understood that this disclosure may include multiple inventive concepts. Use of the singular is for convenience and is not an admission that there is only a single invention.

Mobile Device: In this disclosure, “mobile device” may refer to any portable computing or communication device unless a more specific context is given. This includes, for example, smartphones, tablet devices, smart wristbands or watches, portable projectors, portable speakers, and protective cases for such devices. The protective case may include means to secure the device (such as straps, a handle or hanger, belt clips, etc.) so that the mobile device can be kept in place near the patient. The mobile device (with or without such a case) may also serve as the patient's primary communication device in the system.

Patient: Unless the context clearly indicates otherwise, “patient” refers to the individual who is the focus of the communication system. This is typically a person under care (for example, a hospital patient or nursing home resident) using the invention to communicate with others. However, the term can also include someone in a similar situation of isolation or care (such as a homebound person, an individual in quarantine or protective isolation, or even an animal under veterinary care) who benefits from remote communication with caregivers or family.

Proxy: The patient's medical proxy or designated representative who has the authority to make care decisions and manage communications on behalf of the patient. In the context of this system, the proxy (often a close family member or legally authorized person) may receive clinician messages intended for the family, and generally helps moderate or control the patient's communication channel in the patient's best interest.

Voice Recordings: Voice recordings are a primary medium of communication in this system. Unless otherwise specified, a “voice recording” can include any audio message, not just spoken words. This encompasses singing, natural sounds (like a human heartbeat or breathing sounds, or a pet's sounds), and other audio content meant to comfort or inform. For example, a family member's recording might include them singing a lullaby or a recording of familiar ambient sounds from home for the patient.

Recording: The term “recording” in this disclosure generally means any audio or video data that has been captured and stored (even temporarily) for transmission or playback. A recording may be saved on a device or in cloud storage either before being sent or after being received, or both. It should be understood that a “recording” does not require permanent storage; for instance, a live voice message stream that is buffered for a few seconds before playback is still a recording in this sense. In other words, recordings may be fixed in a non-transitory medium prior to transmission and/or after transmission (or both), or they may be only transiently held in memory during communication.

### DETAILED DESCRIPTION

For simplicity and clarity of illustration, reference numerals may be repeated among the figures to indicate corresponding or analogous elements. Numerous details are set forth to provide an understanding of the examples described herein. The examples may be practiced without these details. In other instances, well-known methods, procedures, and components are not described in detail to avoid obscuring the examples described. The description is not to be considered as limited to the scope of the examples described herein.

It is desirable to provide a communication system that offers features not available in the prior art, such as the ability to record a voice message that plays on a patient's device upon receipt (or is queued for later playback if conditions warrant), the ability to search and filter messages based on text transcriptions, the capability to translate messages into another language and re-synthesize them in the original speaker's voice, and the ability to incorporate indicators of sentiment or emotion in messages (such as tags for admiration, confusion, joy, etc.). The system can play music from a playlist that is set or modified by a family member and sourced from a music streaming service. When connected to a video projector or similar display, the system can show pictures or other images sent by a family member. In one aspect, the patient's device itself could be capable of projecting an image, or it can be connected to augmented reality (AR) or VR glasses/goggles to show content to the patient. In another aspect, the patient's communication device (or its protective case) can be made of materials that allow it to be easily sterilized (for example, a waterproof or antimicrobial case that can be wiped with cleaning solutions or exposed to ultraviolet light without damage). The case may also be constructed from biodegradable materials. In yet another aspect, the patient's device (with its case) can be integrated into a movable robot or robotic arm that is capable of approaching the patient at their request, thereby physically bringing the communication device closer to the patient when needed. These various aspects can be combined in software, hardware, or a mixture of both to form the comprehensive system described herein.

In one aspect, the present invention includes both an application (software) and a companion device or devices that together enable remote family care across distance and visitation barriers. The app is a lifeline that brings together patients, family, friends, and clinicians through heartfelt communication solutions for those facing loneliness and isolation. Unless the context clearly requires otherwise, the term “app” as used herein can refer to some or all components of the invention, whether implemented in software, hardware, or any combination. The app empowers patients, families, friends, and clinicians all to be involved in improving the health and emotional well-being of loved ones. Aspects of the inventions strike a careful balance: caring but not intrusive, innovative but not overwhelming, understanding but not overbearing, and forward-thinking yet grounded in purpose. The technology is aimed at reducing loneliness, isolation, depression, and delirium through an essential yet user-friendly solution. The overarching mission is to encourage hope and curiosity by extending our reach to anyone seeking comfort and emotional connection; in essence, to offer a lifeline during challenging circumstances, providing support and encouragement when it matters most.

### Security and Privacy

To enhance the security and confidentiality of communications, the system employs robust encryption and privacy safeguards. All voice and data communications can be encrypted end-to-end, meaning data is transformed into an unreadable format at the source and only decrypted at the final destination (such that even intermediate servers or networks cannot read it). Various encryption techniques may be used in different embodiments, including symmetric encryption (using a shared secret key for fast encryption/decryption of data) and asymmetric encryption (using public/private key pairs so that no pre-shared secret is required). A hybrid approach may combine these to benefit from each (for example, using symmetric encryption for bulk data transfer and asymmetric encryption for secure exchange of the symmetric keys). Additional security measures can be implemented to reinforce the encryption framework, for instance:

### Secure Key Storage

Encryption keys can be stored in secure hardware or software modules (such as Hardware Security Modules or Trusted Platform Modules) to protect them from unauthorized access or tampering.

### Key Rotation

Encryption keys can be updated regularly, limiting the duration for which any single key remains valid. Automated key rotation schedules help ensure that long-term key compromise is prevented by issuing new keys periodically.

### Standards Compliance

The system adheres to established cryptographic standards and best practices (e.g., NIST or ISO standards) to utilize proven algorithms and protocols. This helps avoid weak encryption and ensures interoperability and reliability of the security mechanisms.

### End-to-End Encryption

As noted, data is preferably encrypted at the source (for example, on the clinician's phone or family member's device) and only decrypted at the final destination (the patient's device), remaining encrypted during transmission and while stored on intermediate servers. This protects data throughout its lifecycle.

### Multi-Factor Authentication (MFA)

Access to sensitive functions (like retrieving certain messages or decrypting data) may require multiple forms of verification, such as a password/PIN plus a biometric check or a one-time code. This adds an extra layer of security to prevent unauthorized access to the communication channel or stored messages.

### Integrity Checks

The system can use cryptographic hash functions and digital signatures to verify that messages have not been altered in transit. Each message or transcript may carry a digital signature or checksum that ensures any tampering would be detected, thereby guaranteeing the authenticity of the content.

Taken together, these comprehensive security measures help ensure that the confidentiality, integrity, and availability of data in the system are maintained. Sensitive information exchanged between patients, families, and clinicians is safeguarded against eavesdropping, unauthorized access, or other potential threats. In addition to encryption, privacy-preserving techniques may be employed. For example, if video data is used, the system can perform real-time de-identification such as blurring a patient's face or removing personal identifiers in a video stream if those are not necessary for the communication. Audio data could be filtered to obscure the patient's actual voice when appropriate (to protect identity) or to eliminate any background information that shouldn't be shared. Patient data that is stored or transmitted can also be stripped of personally identifiable information (PII) where possible. All stored data (whether on the device or on a server) can likewise be encrypted and kept in HIPAA-compliant storage locations. In some implementations, the device might use only local (on-device) storage for recordings to maximize privacy, or use a privacy-focused cloud infrastructure if remote storage is needed.

### System Architecture Overview

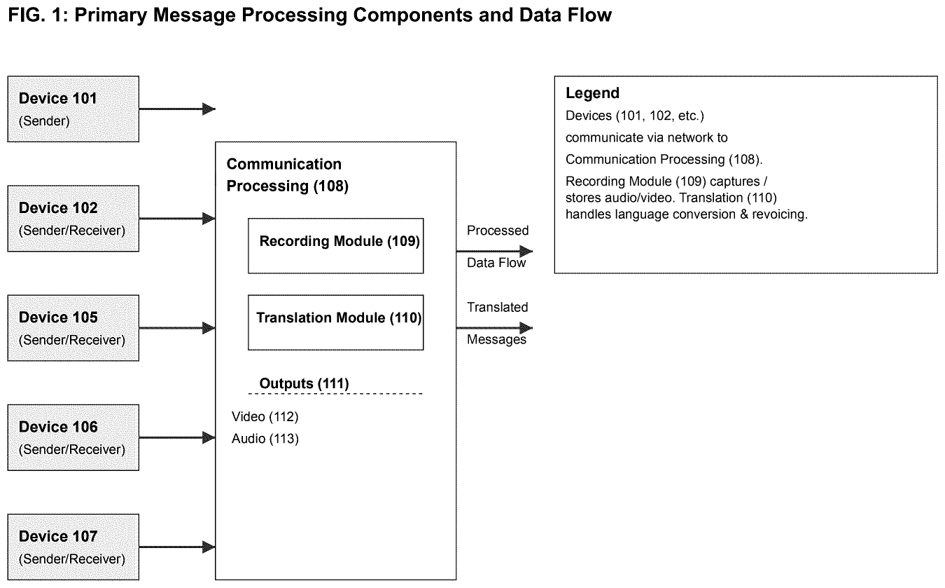

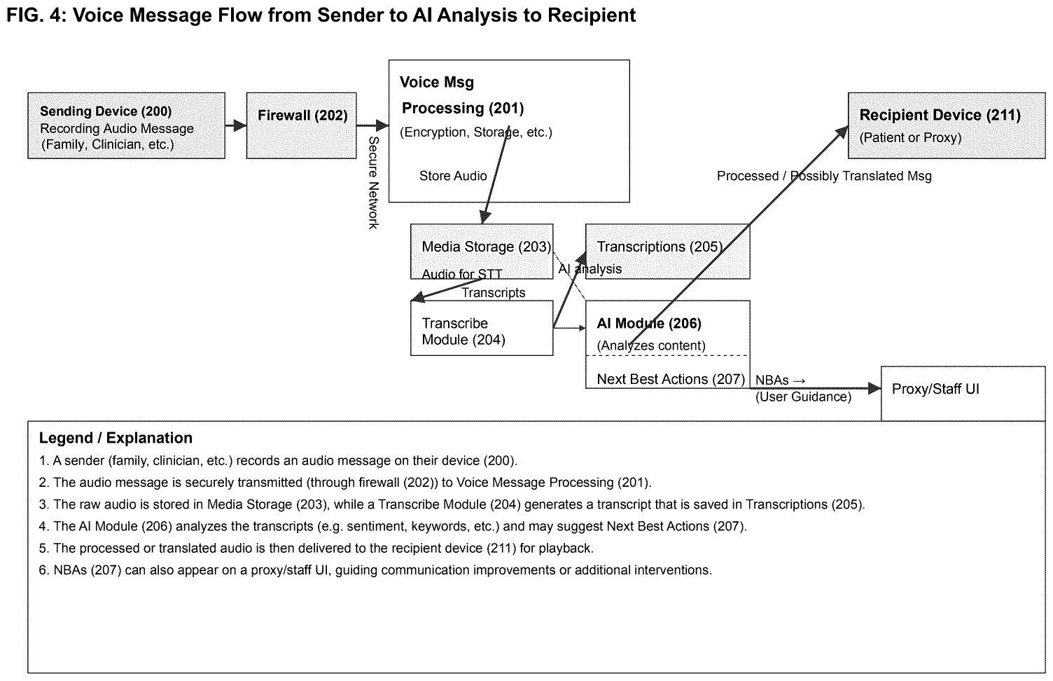

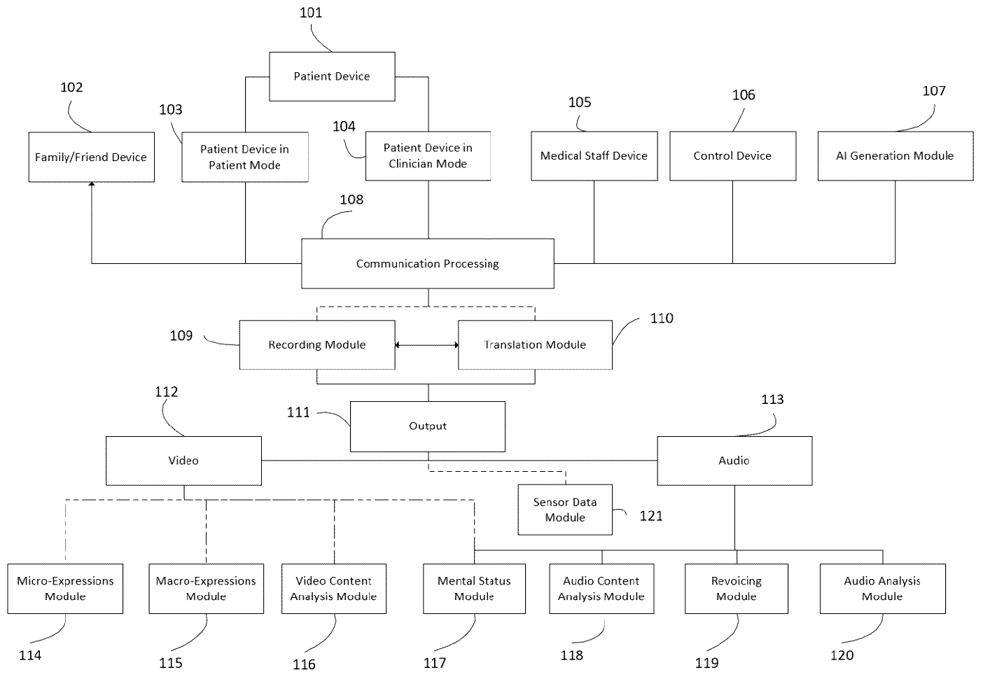

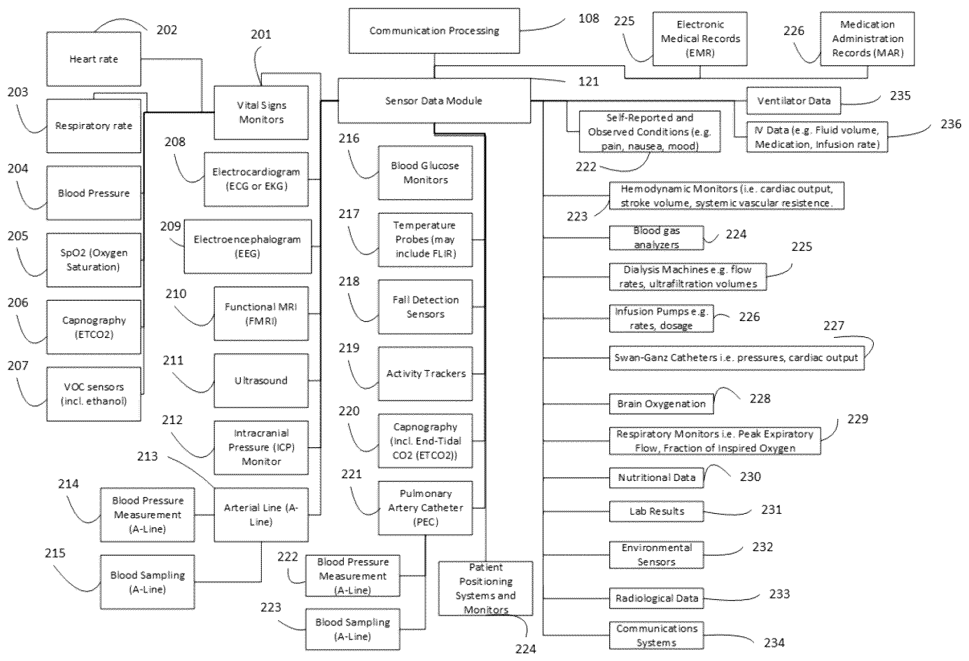

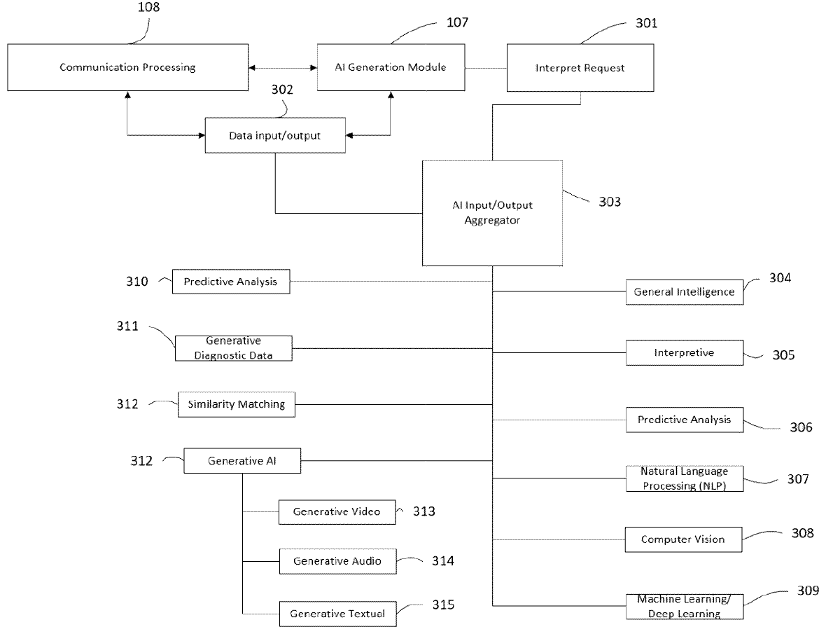

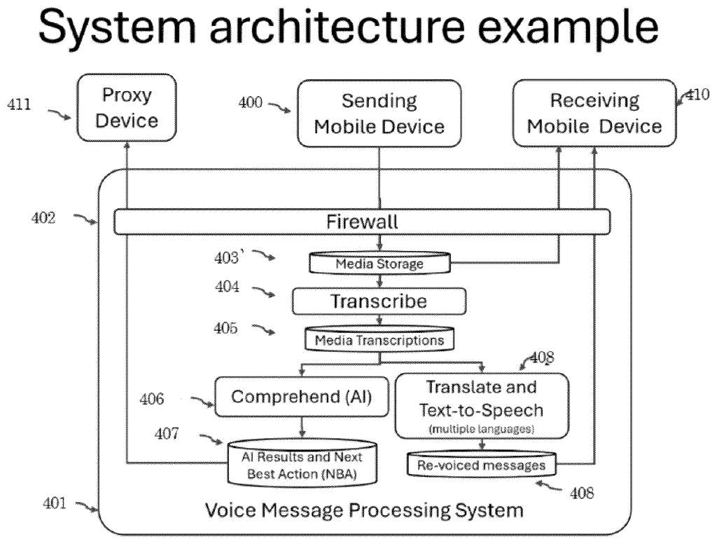

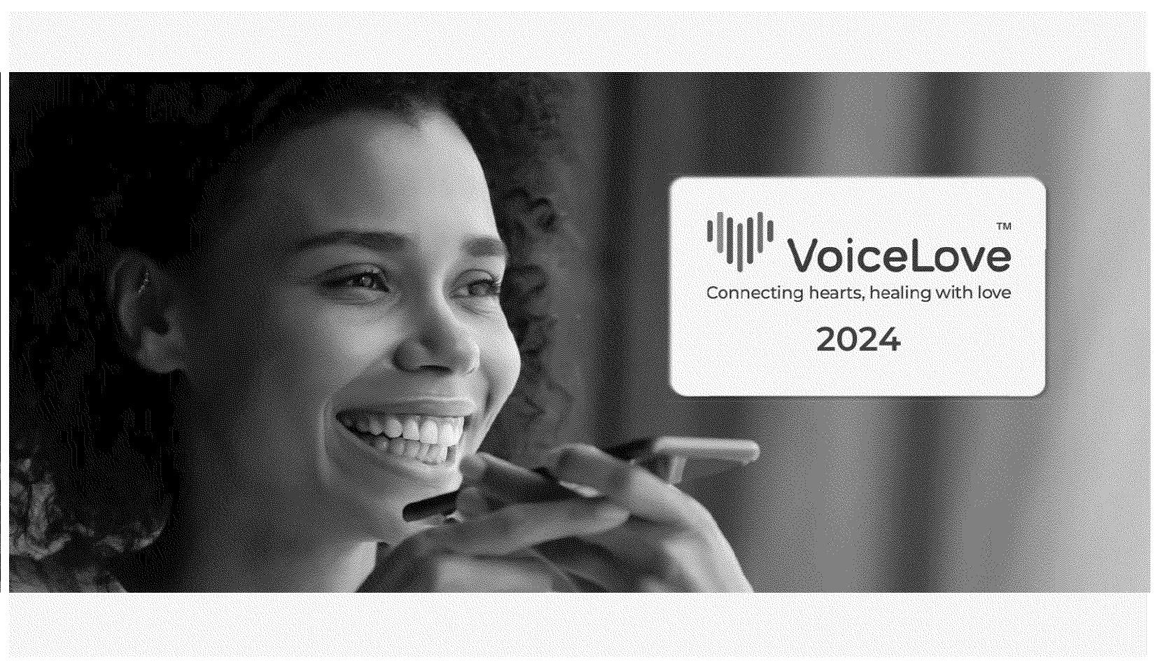

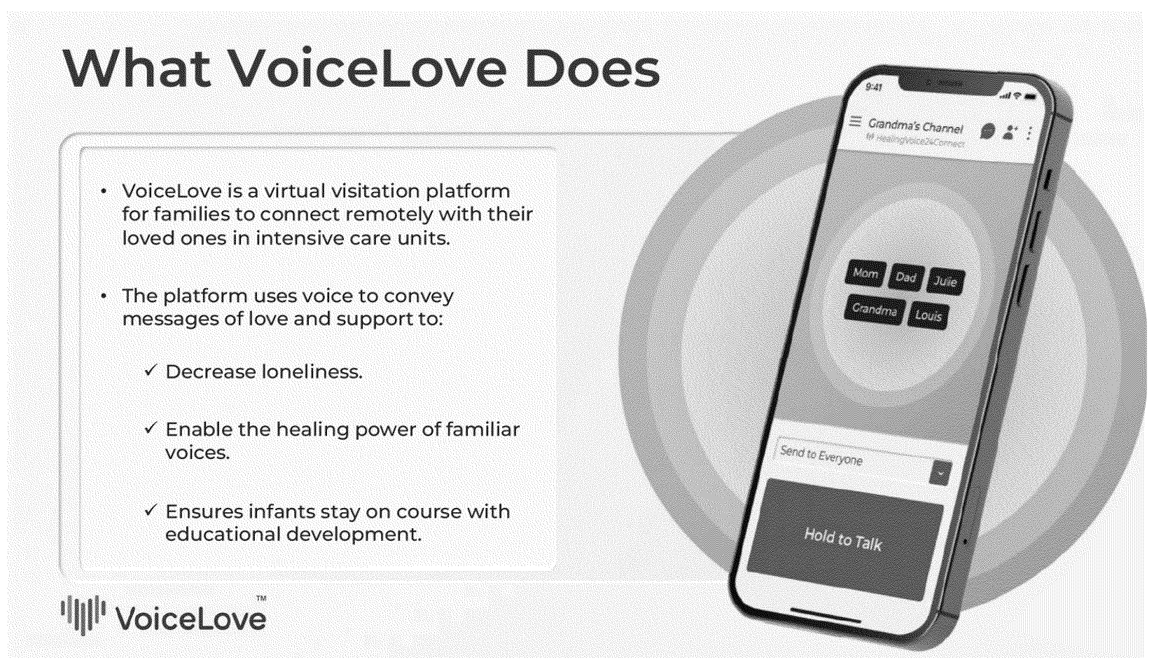

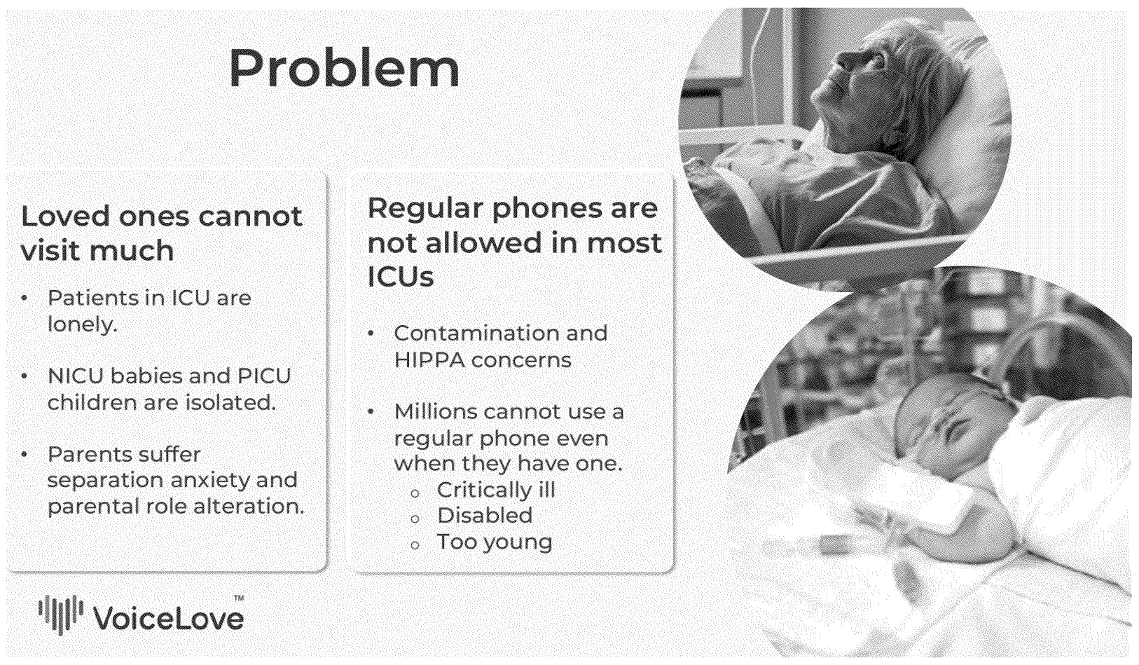

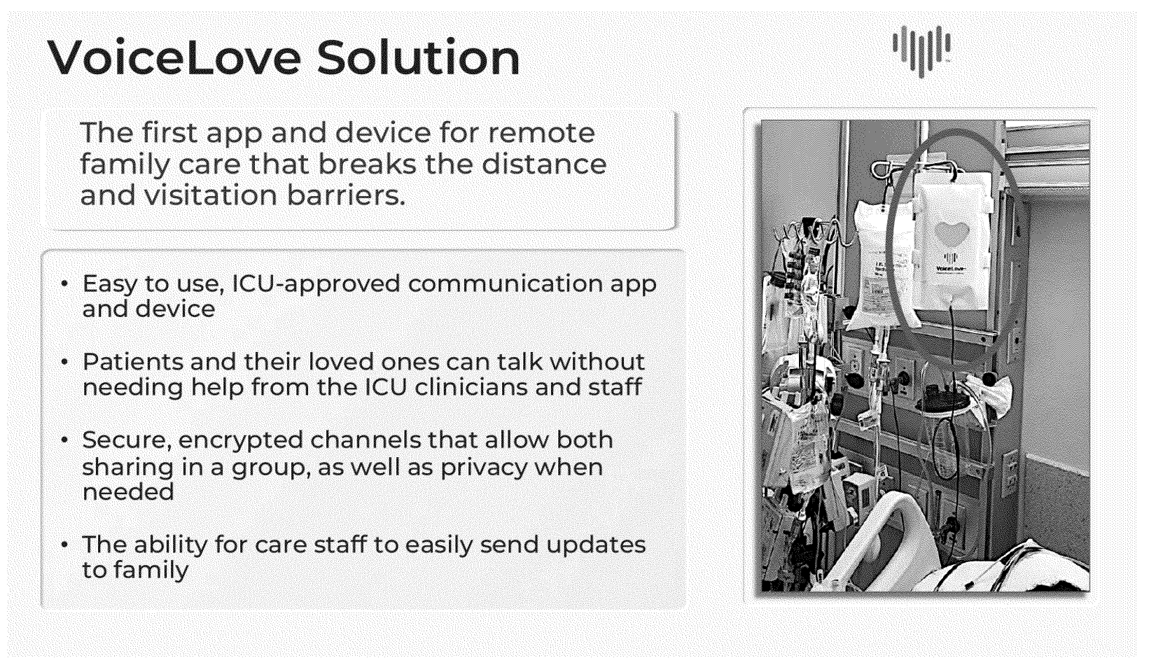

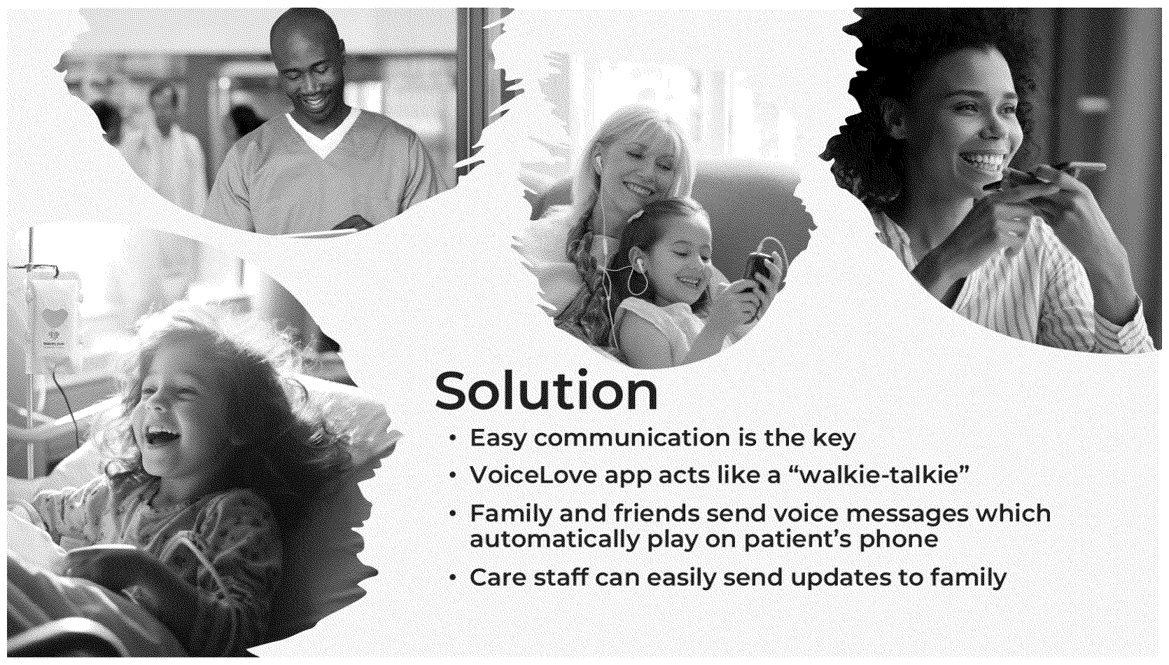

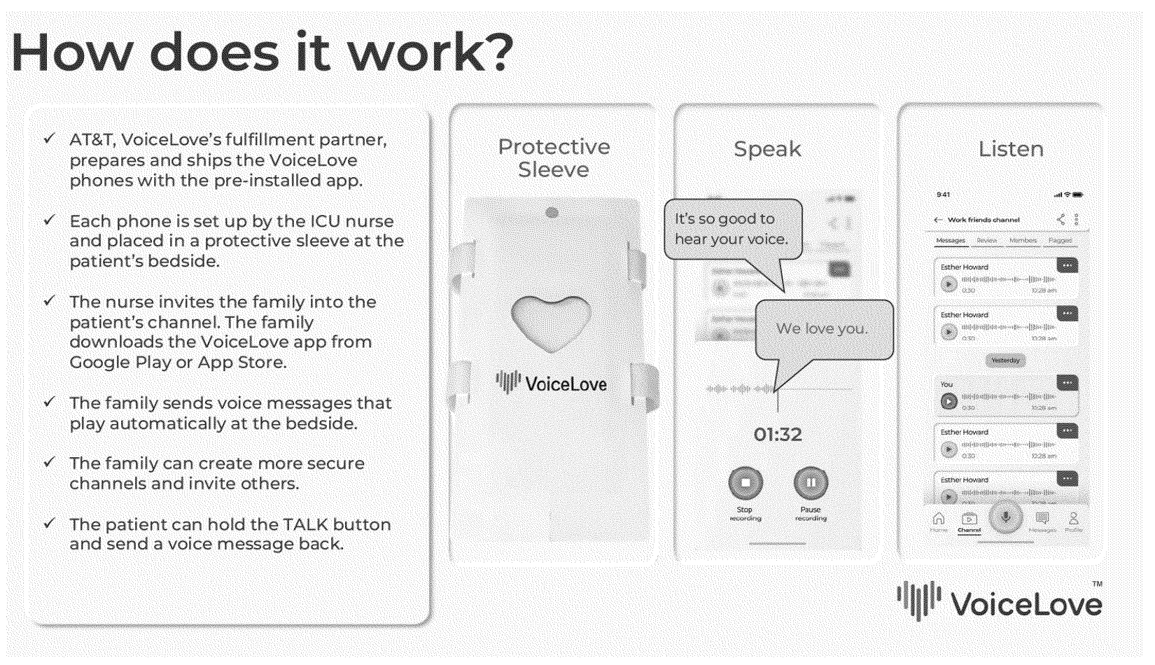

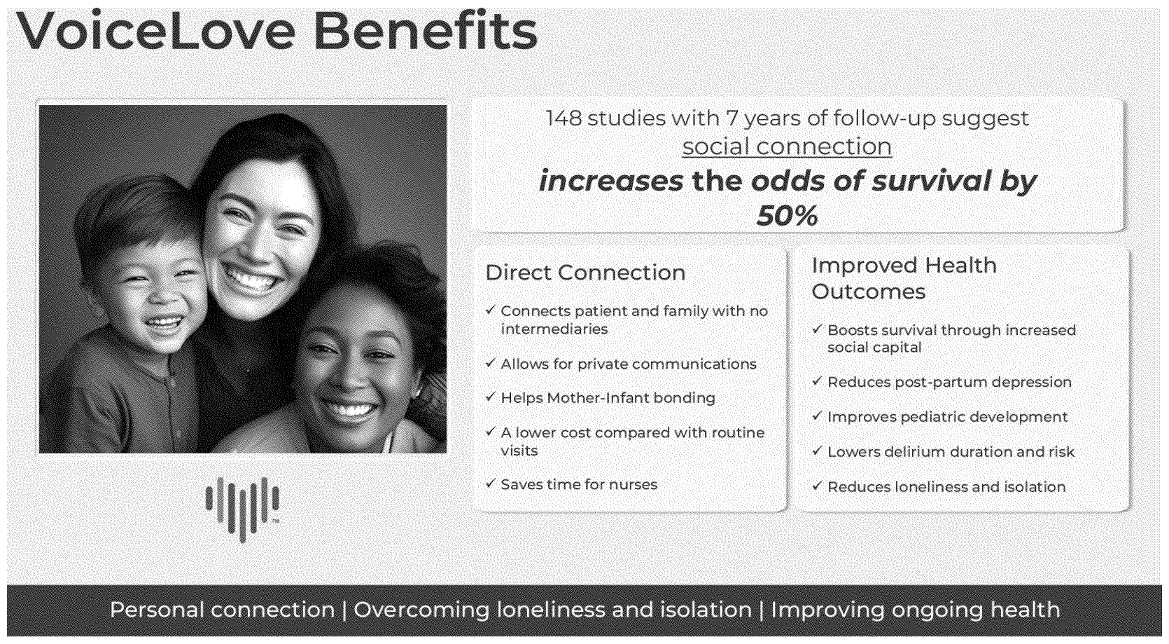

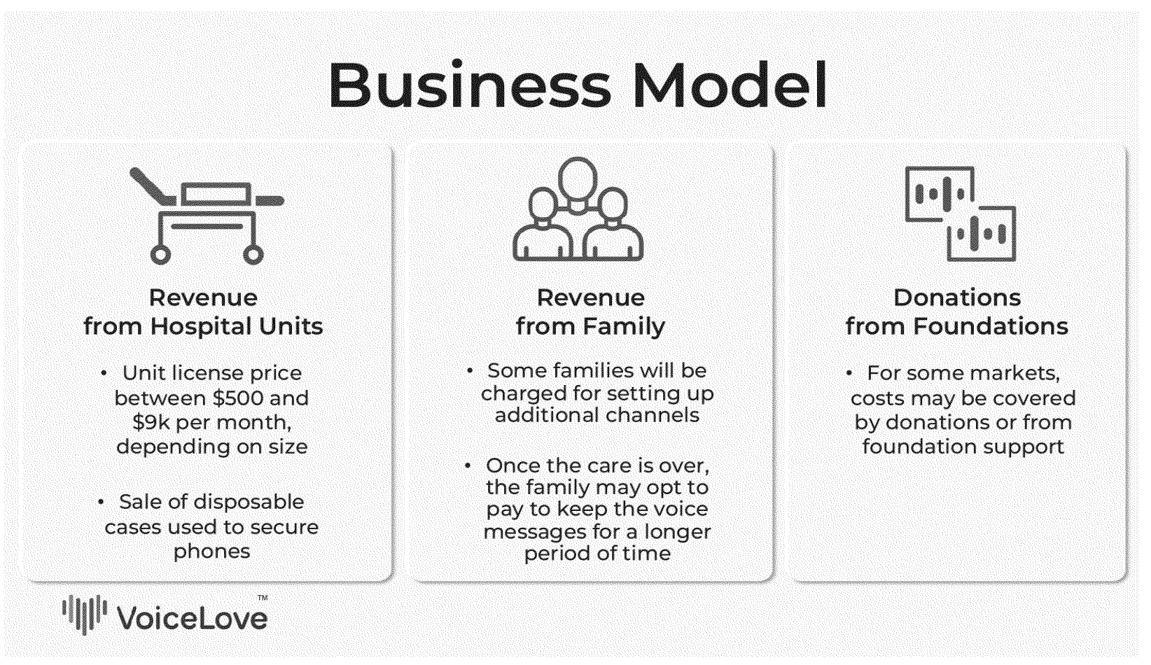

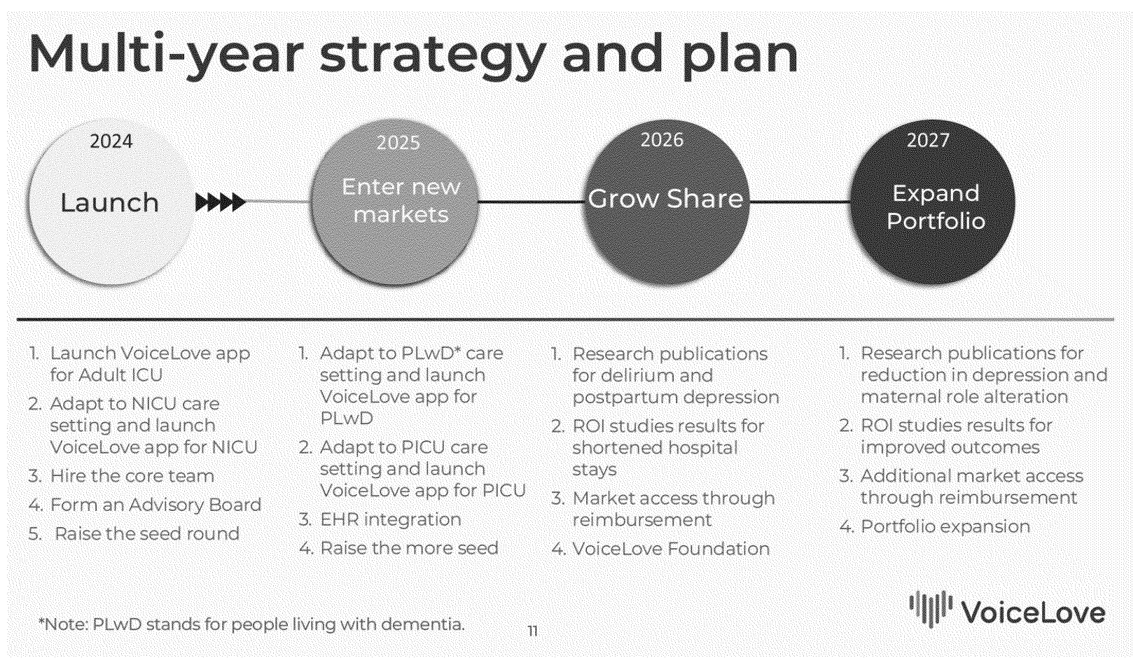

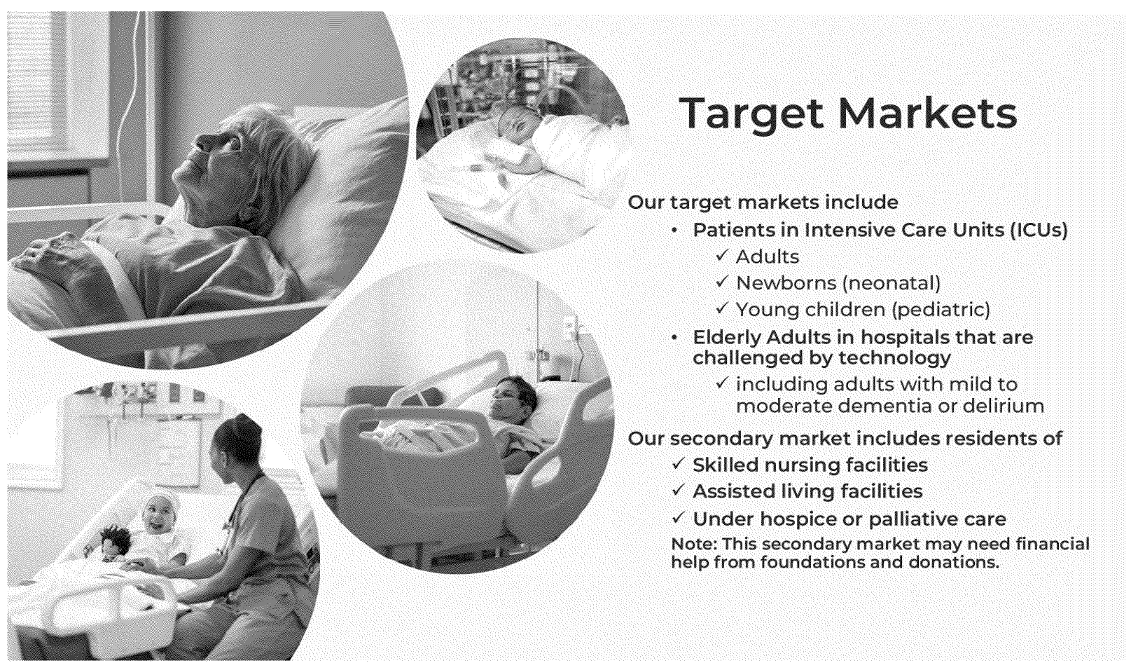

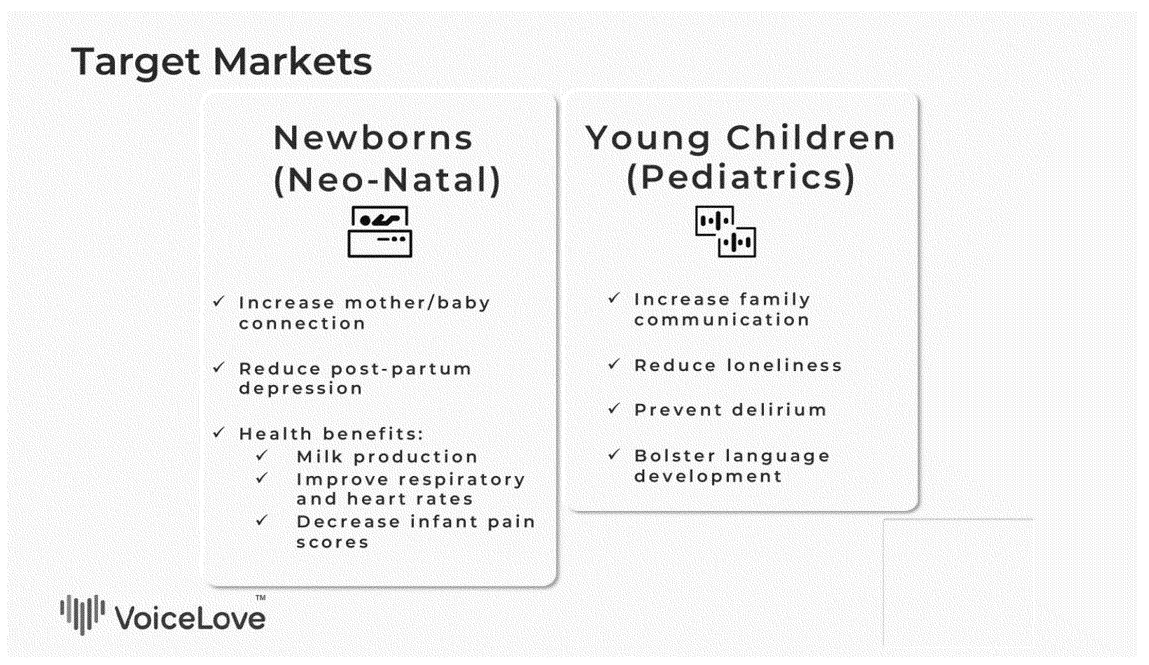

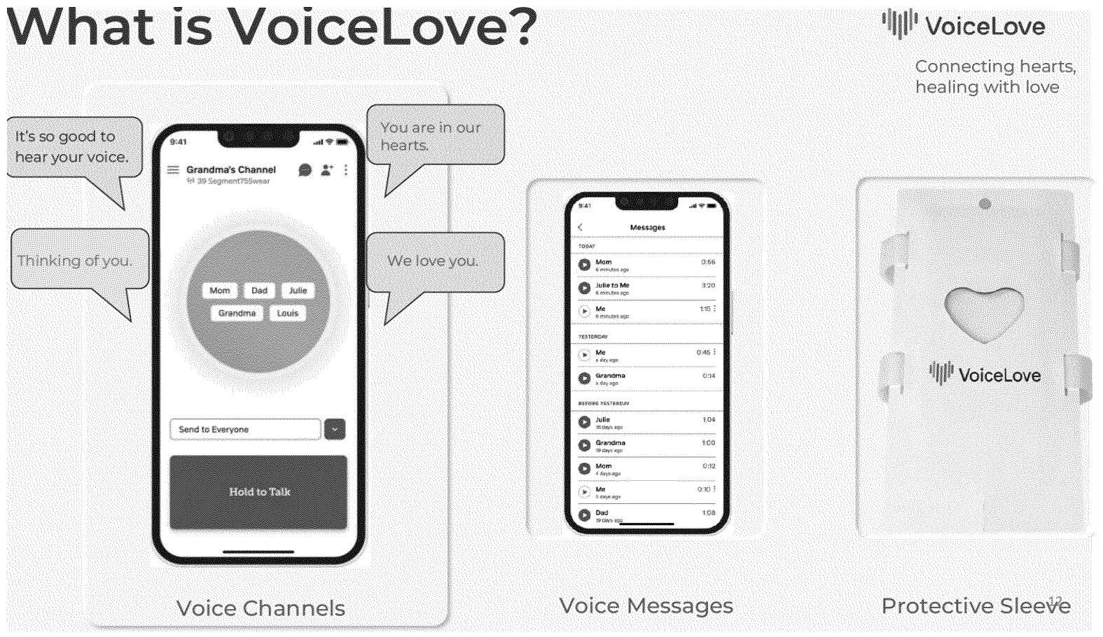

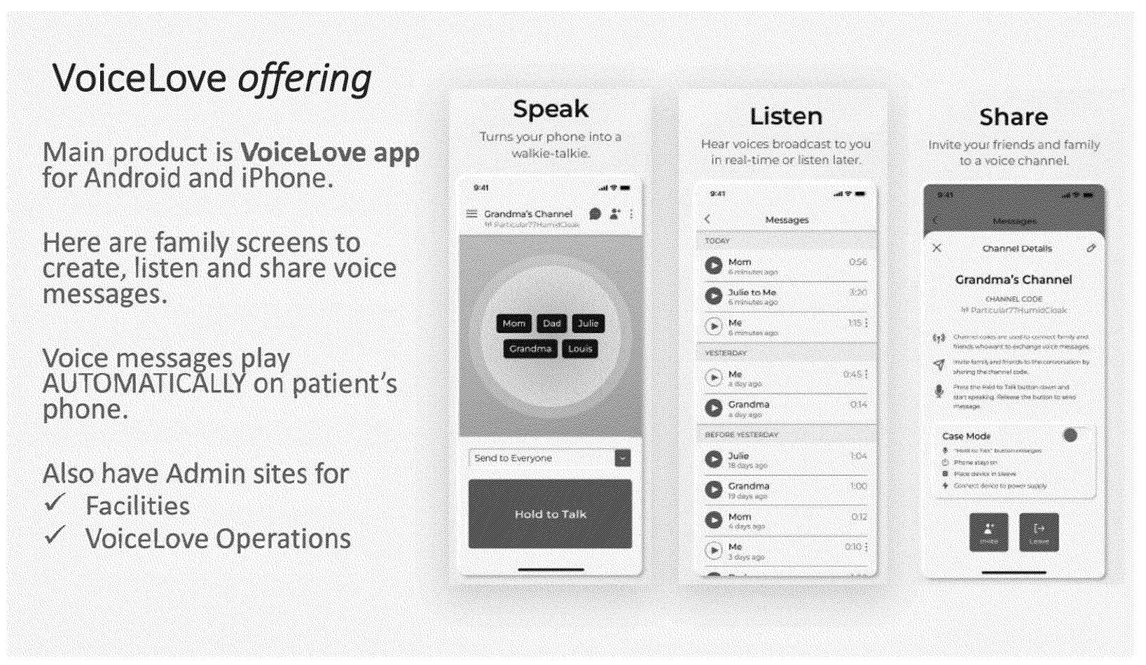

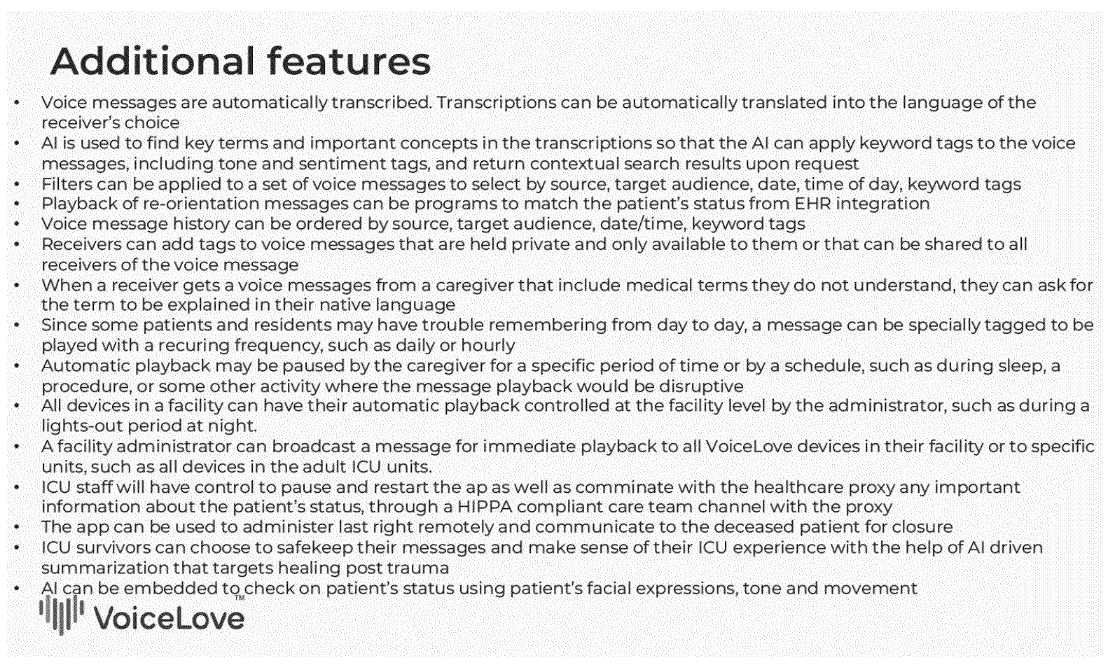

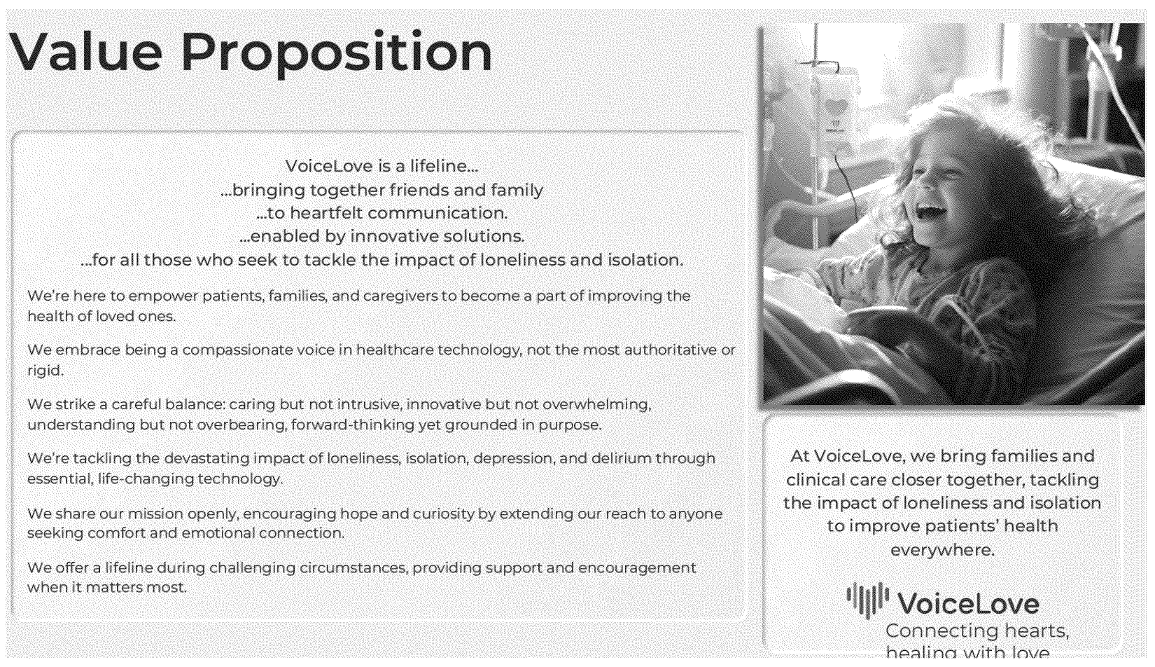

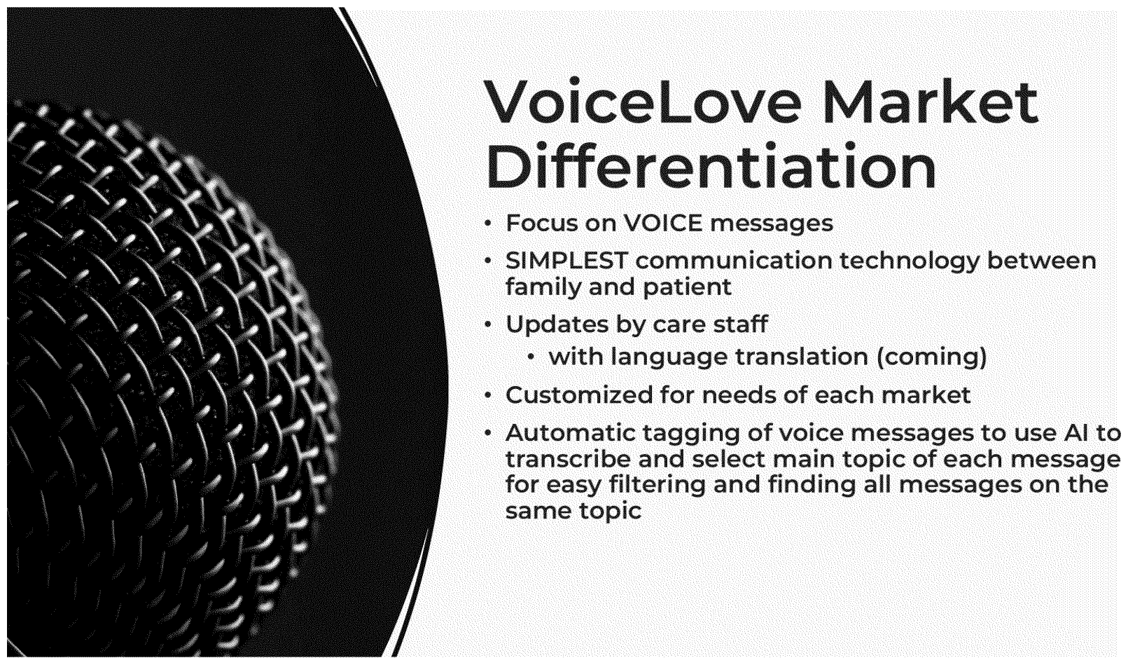

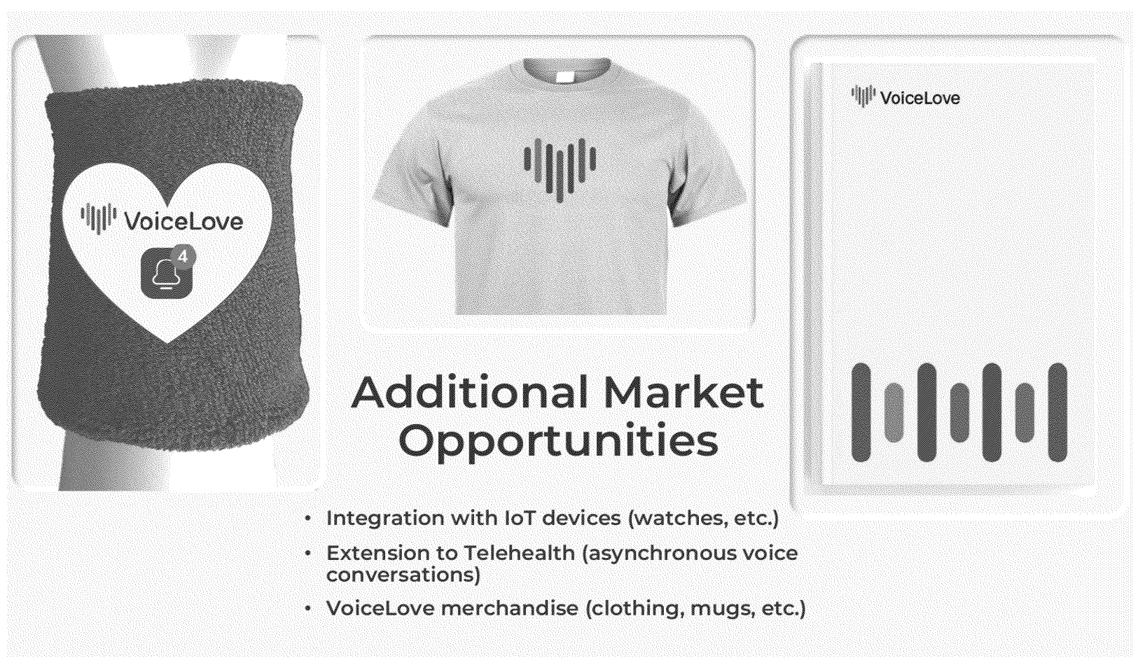

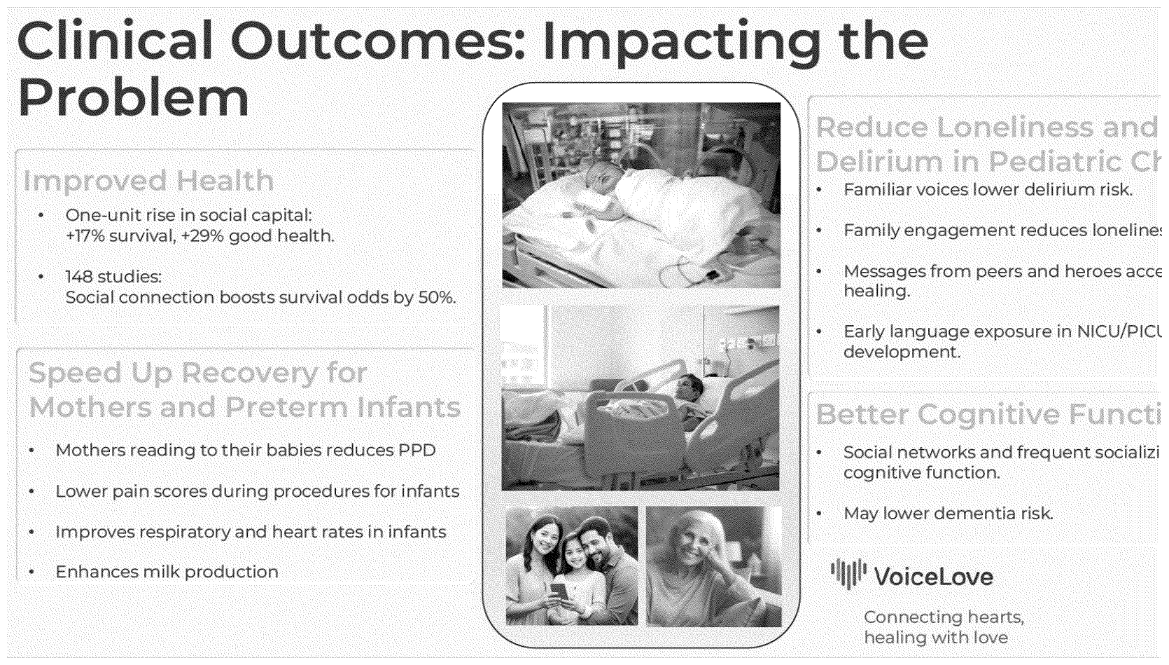

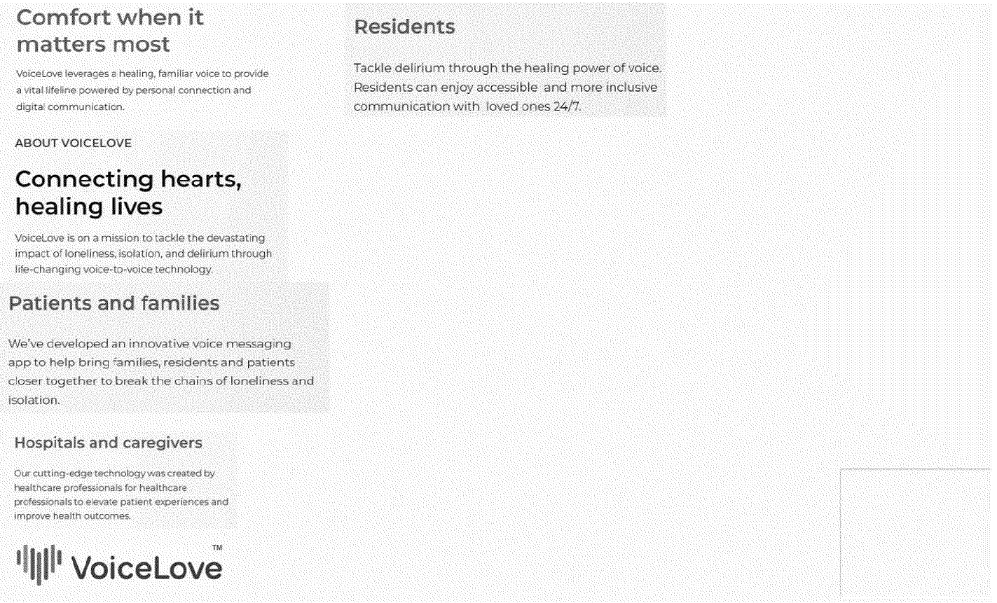

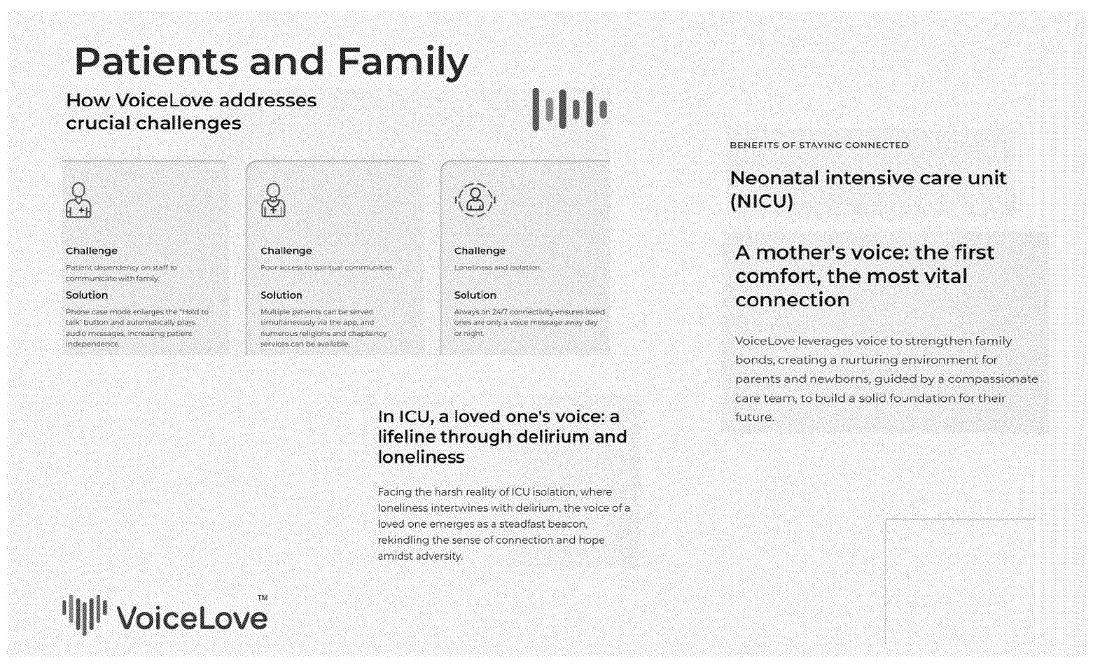

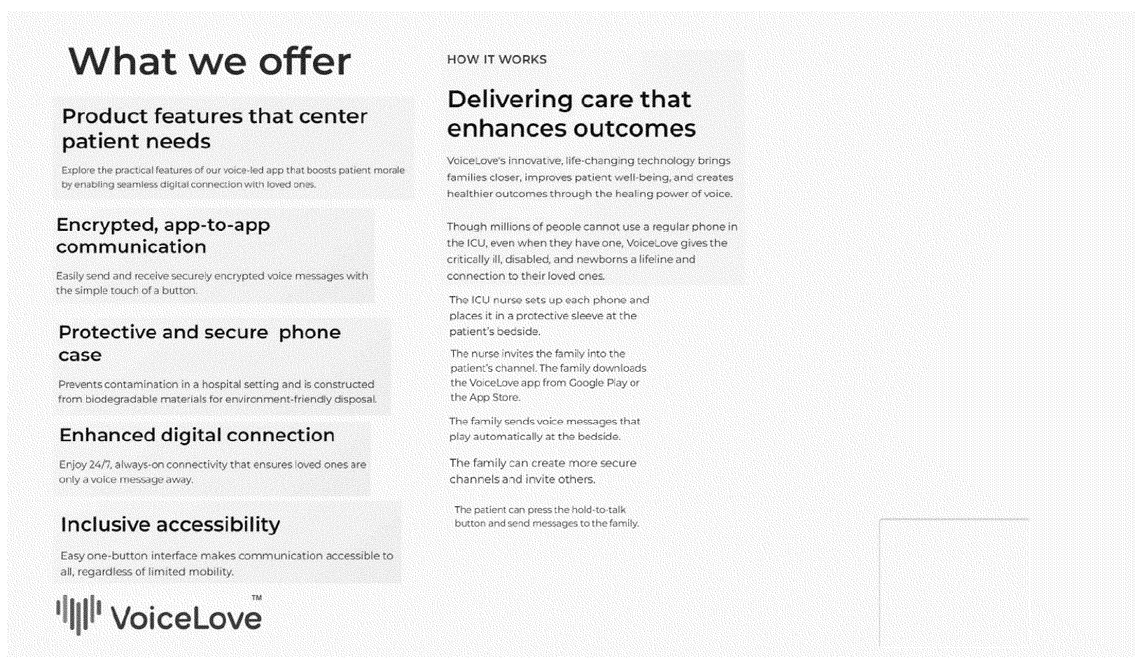

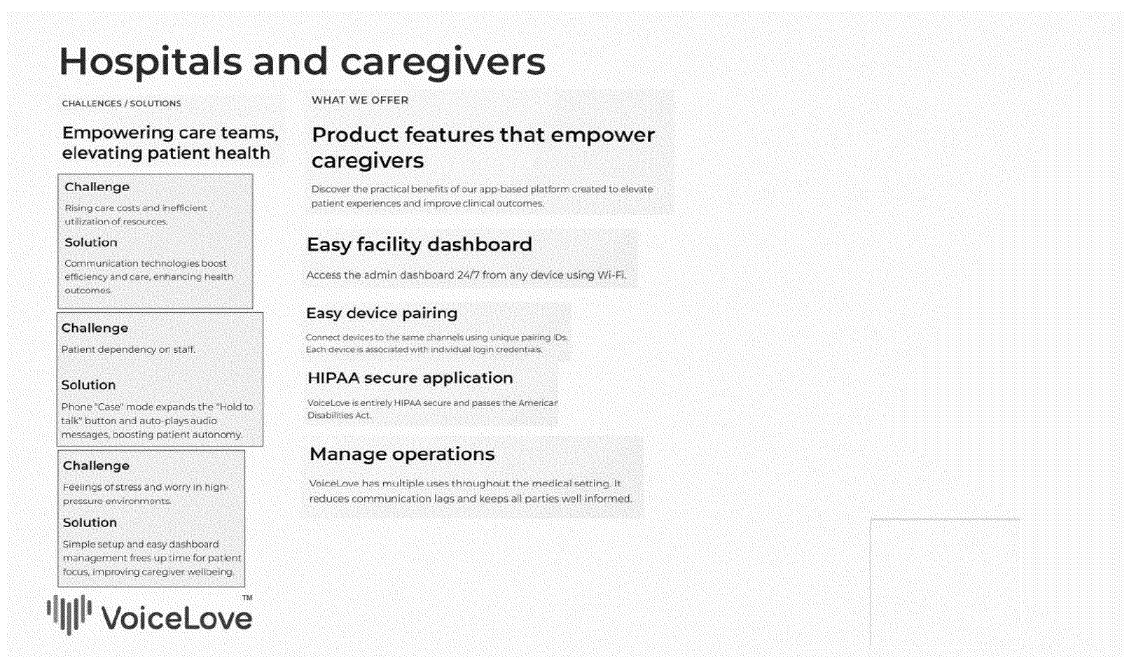

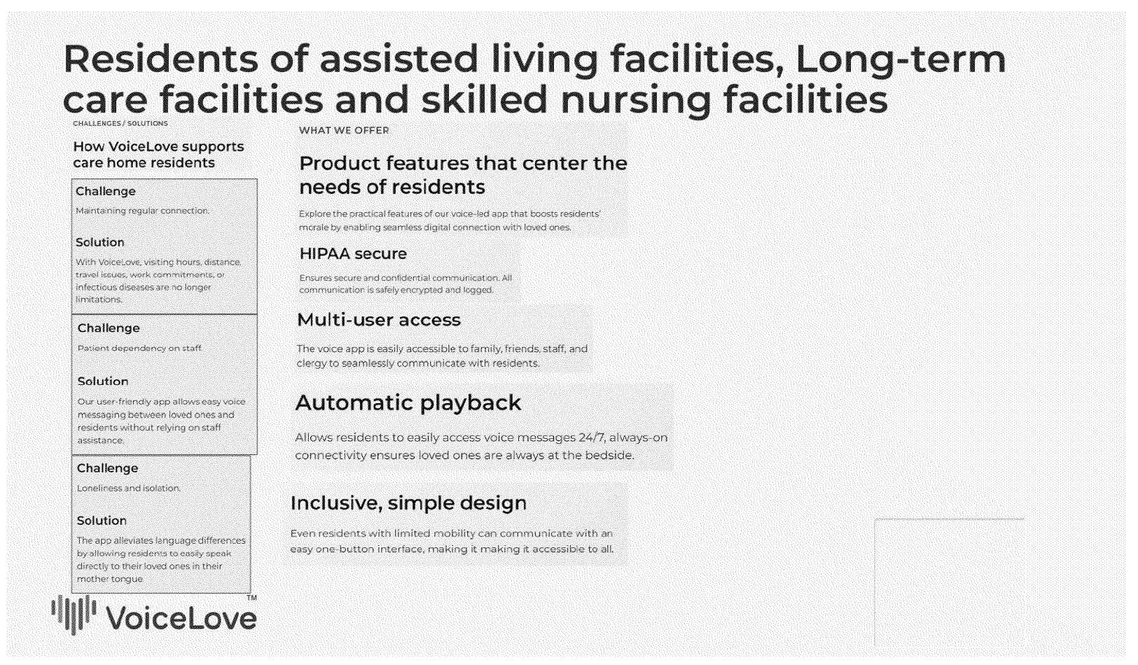

Referring now to an example system architecture (see FIG. 4), the communication system may involve multiple components working together. A patient device (101) is used by or near the patient. A family/friend device (102) is used by family members or friends to send messages. There may also be a medical staff device (105) (such as a nurse's station computer or a clinician's smartphone) operating in a clinician mode for sending updates to the family. All of these devices connect through a communication processing component (108), which may be a cloud-based server system or a set of services (an “app backend”) that handles message routing, data processing, and storage. The communication processing system interfaces with several sub-modules: for instance, a recording module (109) that handles incoming recordings, a transcription module (110) that converts audio to text, and a translation module (111) that handles language translation. Processed outputs (such as translated text or synthesized audio) are then sent to an output module (112) which delivers the appropriate format (audio, video, etc.) to the target device (e.g., the patient's device). Additionally, one or more sensor data modules (121) can feed data from sensors (wearables or environmental sensors) into the communication processing component (108) to inform it of the patient's status. FIG. 1 provides a conceptual diagram of such primary processing components and their data flow.

The sending family device (or clinician device) connects to the communication processing system through a secure network (often via an internet connection through a firewall). When a user records a message and sends it, the voice recording is stored in a media storage area (203 in FIG. 4). Immediately, a transcription task is triggered in the transcription module (204), which transcribes the voice into text and stores that text in a transcription storage (205). The system's AI comprehension module (206) may then analyze the transcription for emotional or contextual content, generating results such as detecting emotional cues or suggesting “next best actions” (207) to improve communication. For example, by analyzing messages from the patient and to the patient, the AI might detect that messages with a certain tone are calming the patient, whereas others might be causing agitation. As guidance to the patient's proxy or family, the system could suggest adjustments (e.g., “speak more slowly and calmly” or “perhaps play soothing music”). It can also flag messages that appear to have a negative impact on the patient's mood so the proxy can review them. These analytic and feedback features are optional but illustrate the deeper integration of AI to optimize the communication experience.

The headings and organized structure provided in this description (such as grouping certain functionalities under subheadings) are meant solely for clarity and readability. They do not limit the scope of the disclosed concepts or examples. Features described in one section may be combined with those in other sections in various implementations, as the invention encompasses numerous interacting components and methods.

### Core Communication Functions

The system's primary communication functions include recording messages, delivering (playing) messages to the patient, and various controls around those processes. These functions are generally handled by the app and associated devices automatically once configured, to minimize the need for patient intervention.

### Recording

The app allows authorized users (e.g., a family member, clinician, or the patient themselves) to record voice messages that can be sent to others in the same communication channel. Recording a message can be initiated in different ways depending on the user's ability and the device: by pressing and holding a record button in the app's interface, by a single press of a button (with the app automatically stopping at the appropriate time or using a separate stop button), or by issuing a voice command (hands-free). The app can also be configured to automatically stop recording if it detects a period of silence longer than a predefined threshold, to avoid recording unnecessary silence. For example, if no voice or sound is detected for, say, 30 seconds, the app could assume the message is finished and stop the recording. (The transcription module can assist in detecting when the speaker has stopped talking.) The recorded message is then packaged (optionally after transcription or other processing) and sent through the system to the intended recipient(s).

### Playback

Once a voice recording is sent to a channel, all other participants in that channel (for instance, the patient and other family members on that channel) will have the message delivered to their devices. The patient's device is typically configured to automatically play the voice message as soon as it is received, so that the patient hears the message without needing to touch the device. If the patient's device is currently in a “paused” state (as described below), the message will be received but held without playing until the pause is lifted. In most cases, the patient hears messages in real time or near-real time after someone sends them. To ensure courtesy and minimize disruption, the system can play a short alert tone or chime just before playing an incoming message and introduce a brief delay. This gentle alert gives anyone present in the room (such as a nurse or doctor with the patient) a moment to pause or cancel the playback if the timing is not appropriate (for example, if the patient is in the middle of a medical exam or a sensitive moment). If no one intervenes within those few seconds, the message will then play automatically. The alert tone and delay feature can be configured (or disabled) according to facility policies or user preferences.

### Explanation

The receiver of a message (or an authorized caregiver) can request an explanation of terms in a received message. This is especially useful for medical jargon or technical terms that a family member or even the patient might not understand. For instance, if a clinician's voice message to the family proxy mentions a medical term, the proxy can tap that term in the transcript or issue a voice query to get an explanation. The system can provide a brief definition or explanation of the term. Moreover, the explanation can be delivered in the listener's preferred language. For example, if the proxy's app is set to Spanish, it can display or speak the explanation of an English medical term in Spanish. This cross-linguistic explanation feature leverages the translation capabilities of the system to improve understanding of important information.

### Pause

A user (typically the patient or someone managing the patient's device) can pause the app, which temporarily stops it from automatically playing new incoming messages. When the app is paused on the patient's device, any messages that arrive will be queued but not played until the patient (or a caregiver) unpauses the device. Pausing might be useful during times when the patient needs rest or during nursing care routines. Additionally, facility administrators or clinicians can issue a pause command for multiple devices in a ward or entire facility. For example, in a hospital unit, all patient devices could be paused during the night or during certain quiet hours. The app supports scheduling a recurring pause period (such as automatically pausing every night at 10 PM and resuming at 7 AM). If a message sender (like a family member) sends a message while the patient's device is paused, the system can indicate to the sender that the message will be delayed (for instance, the sender's app may show a small icon or note that the recipient is currently in “Do Not Disturb” mode). The sender may also be able to see when the pause is scheduled to end (e.g., “paused until 7:00 AM”). This way, all channel members are aware of the communication status. When the patient's device is unpaused or the scheduled pause period ends, any queued messages will automatically play in the order they were received. The system also supports a facility-wide pause control: an authorized staff member using an administrative console can pause all devices in a particular unit, department, floor, or the entire facility (for example, initiating a “quiet hour” at night for all patients). The console can also broadcast a short audio announcement (like “The system is entering quiet mode”) to all devices when such a facility-wide pause is activated, so that anyone present knows why messages are not playing.

### Reorientation Messages

The patient's proxy or caregiver can create a special kind of message designated as a reorientation message. A reorientation message is intended to help reorient a patient who may be confused (for example, patients suffering from delirium or dementia, or waking from sedation). Such a message typically reminds the patient of where they are, what is happening, and reassures them. The system marks these messages distinctly from regular messages so they can be easily found and played. A reorientation message can also be set to play repeatedly at scheduled intervals (e.g., every hour or every morning) to continually remind the patient of their situation, if needed. For example, an authorized user could schedule a reorientation message like “Hello John, you are in the hospital recovering from surgery. You are doing well and we are taking care of you.” to play every morning at 8 AM and again in the afternoon, etc. These recurrence settings are configurable.

### Multi-User Channels

The app supports multiple communication channels and group messaging in a way that is user-friendly even for non-technical users. Multiple groups of people can be connected with the patient through different channels. For instance, the patient might have one channel for immediate family, another for extended family or friends, and another for a religious community or support group. The patient (or proxy) can thus control who receives what messages by choosing the appropriate channel. The system's translation capability means that even if a channel includes members who speak different languages, the voice messages can be automatically translated per recipient as needed. For example, if a patient sends a single voice message to a channel that includes English-speaking and French-speaking relatives, the English speakers can hear the message in English while the French speakers receive it in French, thanks to automatic transcription and translation on the fly. Any user can create a new channel (for example, a family member might set up a new channel and invite additional relatives via the app, sending them an email or text link to join). Users can belong to multiple channels for one patient, and channels can be archived or closed as needed. In institutional settings like long-term care facilities, the initial private channel for a patient is typically set up by clinical staff to ensure that only an authorized family member (proxy) is connected at first. Thereafter, the proxy can invite others. If a channel needs to be terminated (for example, if the patient is discharged or moved), the system can allow the proxy or family to download the history of voice messages from that channel for safekeeping. When a channel is shut down, the server may retain its message history for a grace period (e.g., a few weeks) to allow time for the data to be saved offline by the family.

### Search and Filter

All messages that are recorded are automatically transcribed to text, as noted. This enables a powerful search function in the app. A user can search through past voice messages by keyword or apply filters to find specific communications. For example, a family member might search for all messages where the patient said the word “pain” or look up all messages sent by a particular relative. Filters can be applied by message source (who sent it), by target audience or channel, by date or time range, by tags, or by detected sentiment. For instance, the user could filter to see “messages from Mom last week” or “messages tagged as ‘love” or “all messages in January where the AI detected a sad tone.” Search results can return both the text transcript and allow playback of the original audio. This feature benefits clinicians and family alike-clinicians could quickly review what interactions have occurred (through text summaries) and family members could recall what updates were given.

### Broadcast Messaging

In a facility setting, authorized personnel can broadcast messages to multiple patients at once. For example, a hospital administrator could use the system to send an announcement that will play on all patient devices in a certain area. The app's administrative console might have an option to broadcast to “all devices on Floor 5” or “all ICU devices” etc. When such a broadcast is sent, each target device will play the message (subject to any pause mode or quiet hours in effect). An example use might be a nightly announcement: “It's 10 PM, we are dimming the lights for quiet hours. You can still listen to saved messages, but new family messages will be held until morning.” This ensures consistent communication across the facility. Broadcast messages can be immediate or scheduled (like a recurring nightly reminder). Additionally, a facility administrator can choose subsets—for instance, broadcast only to devices in a specific wing or to all devices assigned to a particular unit type.

### Rich Media and Additional Capabilities

Beyond basic voice messaging, the system encompasses various features to enrich the communication experience and tailor it to patient needs and preferences:

### Music Streaming

The app can play music for the patient, either from a predefined playlist or by genre/station. Uniquely, a remote user (such as a family member) can direct what music is playing on the patient's device. While streaming music to a device is not inherently novel, giving remote loved ones the ability to select or change the patient's music in real time provides a new way to comfort the patient. For example, a daughter at home could pick her father's favorite soothing songs to play for him in the hospital at bedtime. The patient or clinician can always override or pause the music if needed.

### Picture Slideshow

Similarly, the system can display images on the patient's device or an attached screen. A family member could upload pictures (such as family photos or uplifting images), and the app on the patient's device will show these as a slideshow. If the device is connected to an external projector or large display (wirelessly or via cable), it can project these images onto a wall or screen in the patient's room. The novelty here is not slideshows per se, but the ability for remote users to curate and control the content shown to the patient. It's a way for family to personalize the patient's environment from afar (for instance, updating the slideshow with new pictures of grandchildren).

### Memory and Event Recording

In some embodiments, the system may continuously or periodically record aspects of the patient's environment to help with memory or clinical review. For patients prone to memory impairment, paranoia, or hallucinations, it can be helpful to have a factual log of events. The device may use its camera and microphone (or other sensors) to capture what happens around the patient (subject to privacy settings and legal considerations). This could be done at set intervals or triggered by events (motion, sound, etc.). Using computer vision and/or audio analysis, the system can then summarize these events in a human-readable form. For example, the system might note: “Patient was visited by Person A from 1:30 PM to 1:42 PM,” or “At 10:22, patient sat up in bed. At 10:23, patient's eyes closed and patient slumped over; at 10:24, patient was on the ground; patient moved again at 10:27.” This kind of automated log can be extremely useful diagnostically (the latter example might indicate a fainting or fall event). The data can be stored temporarily on the device and be accessible to medical staff for review. External sensors such as room cameras could be integrated for this purpose as well. In some implementations, sensors like LIDAR (for precise motion tracking) or thermal cameras (to monitor presence or temperature without revealing identities) might be used to capture events while preserving some privacy (for instance, thermal imaging can show that a person was present and moving without providing a clear visual identity). These recordings or logs can help staff see what occurred in their absence (subject to patient consent and privacy rules).

### External Sensor Integration

The device or system can integrate a variety of external sensors to enhance functionality. These sensors can be built into the patient's device, worn by the patient, or placed in the environment. Examples include: vital sign monitors (heart rate, blood oxygen (SpO₂), blood pressure, respiratory rate), motion detectors or pressure sensors (to detect if the patient is out of bed), and cameras with AI analytics. If these sensors detect certain conditions, the system can respond accordingly. One important use-case is safety monitoring: if a sensor indicates the patient may be in distress or has experienced an accident, the system can generate an alert. For instance, a camera aimed at the patient might use AI to detect a fall. If the patient is seen falling or on the floor, the app can automatically place a phone call or send an urgent notification to the nursing station and/or the patient's proxy, alerting them to check on the patient. Similarly, the system can use hospital telemetry (like an ICU monitor) to determine if the patient is asleep or on certain medications (e.g., high doses of sedatives) and thus should not be disturbed by non-critical messages-effectively implementing a “do not disturb” mode driven by medical data. If that telemetry later indicates the patient is awake or more alert, the system can resume normal message delivery.

### Wearable Device Integration

A specialized case of sensor integration is the use of wearable devices on the patient. In one embodiment, the patient may wear a device on their wrist (such as a smartwatch or a custom wristband) that runs a companion app or sensor suite. This wearable can collect data like movement, heart rate, skin temperature, etc., and transmit that data to the main communication app on the patient's primary device (e.g., a tablet at the bedside). The system can change its behavior based on this data. For example, if the wearable's motion sensors detect that the patient is tossing and turning (perhaps indicating discomfort or an attempt to get up), the system might notify a nurse or at least flag that the patient is active. If the patient's blood oxygen drops below a threshold as measured by a wearable pulse oximeter, the system could pause any non-urgent messages (to avoid aggravating a possibly serious situation) and alert clinical staff. The wearable effectively extends the system's awareness of the patient's state and can also serve as an additional user interface. If the patient cannot reach or operate the main device, they might use the wearable to trigger voice commands or send simple signals. For instance, the wearable could have a one-button interface that the patient presses to start recording a message, or a voice-activation (microphone) to capture a message if the patient says “record.” In another scenario, if the patient lacks the motor control to even press a button, the wearable or device can be configured to respond to minimal motions (like a particular facial gesture or a vocal sound). Thus, for patients with extremely limited mobility, the system can be actuated by a simple gesture (perhaps blinking twice or humming) as detected by a wearable EMG sensor or camera on the device.

### Voice Activation and Hands-Free Control

As touched on earlier, the system can be controlled with voice commands, which is crucial for patients who cannot use their hands reliably. A patient can speak a code phrase (for example, “Hey VoiceLove, send a message”) to wake the system and begin recording a voice message to family. The system may either take that single command and then automatically record whatever the patient says next as the message, or it could engage in a brief dialog to clarify the recipient or content (especially if the patient has multiple channels; the system might ask “Who do you want to send a message to?” and the patient could reply “my family group”). The use of a wake-word like “Hey VoiceLove” (VoiceLove being an example name for the service) allows the patient to trigger recording without physically touching the device. Additionally, specific commands can trigger different functions; for example, the patient could say “Help” to trigger an assistance alert (as mentioned above). If the patient says “help” and does not cancel that request within a configurable period, the system can automatically page a nurse's device and/or send a notification to family members that the patient needs immediate help. This voice-triggered help feature acts as a safety net in case the patient cannot operate a call button. The system is designed to distinguish such keywords and not confuse them with normal conversation (hence the use of a specific wake-word or being in a dedicated listening mode for commands).

### AI-Based Tagging and Sentiment Analysis

All voice messages that pass through the system can be analyzed by AI to extract useful metadata. The AI module can transcribe the audio and then look for key terms, phrases, or tones. It can apply keyword tags to each message (for example, tagging a message with “birthday” if it detects birthday wishes, or “medical update” if certain clinical words are used). It can also infer the emotion or sentiment behind messages—e.g., identifying that a message from a family member is cheerful, or that a patient's voice sounds anxious. Tags like “agitation detected” or “happy tone” can be attached. These tags then feed into the search/filter functionality (so one could filter messages by emotion or topic). They can also trigger actions; for instance, if a message from the patient is tagged as “frustration” the system might alert the proxy to check on the patient's emotional state. The AI can also summarize content if needed, or highlight important parts of messages (for example, if a long message mentions “doctor said you can go home tomorrow,” the system could flag that sentence for the patient or family).

### Cross-Language Translation and Synthesis

A core innovative aspect is the automated cross-linguistic communication. When a voice message is sent, the system can determine the preferred language of the recipient. If the sender's and receiver's languages differ, the transcription module will pass the text to the translation module. The translation module converts the transcript from the original language to the target language. Then, the system uses a voice synthesis engine to generate speech audio of the translated text. Notably, the voice used for synthesis can be chosen to resemble the original speaker. For example, if a grandmother sends a message in English to her grandchild who only understands French, the system can produce a French audio message that still sounds like the grandmother's voice (by using voice cloning technology on the original recording). This adds a personal touch that is often lost in translation. If the receiver does not require translation, the original recorded audio is simply played. The choice of language could be automatically determined by the app's settings or explicitly selected by the receiver. In some cases, the system might send both versions (original and translated) to the receiver, giving an option to switch. This translation feature allows seamless communication in families spread across different language backgrounds. It also helps in clinical situations—for instance, if a doctor who speaks one language is updating a proxy who is more comfortable in another language, there is less chance of misunderstanding.

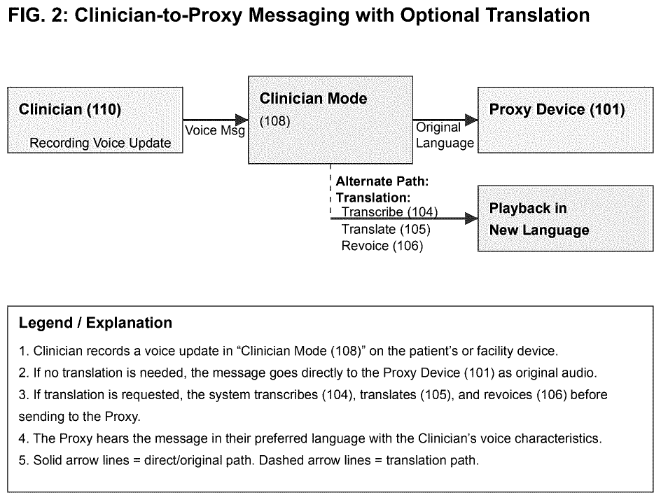

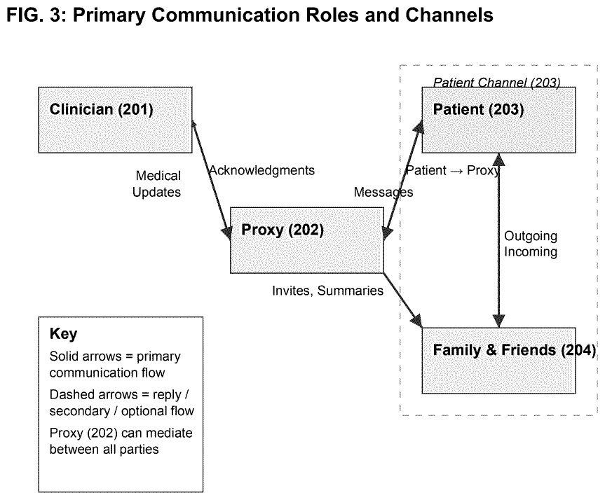

As an illustrative example (see FIG. 2 for reference), consider a clinician (201) at the patient's bedside who wants to send an update to the patient's proxy (202). The clinician uses the patient's device (which is set in “clinician mode”) to record a voice message about the patient. The proxy, who might be remote, has their own device (101) set to their preferred language. If the proxy has chosen to receive messages in the original language, then as soon as the clinician finishes recording and sends the message, the proxy's device will play the clinician's voice message directly. However, if the proxy prefers an alternate language, the system will automatically transcribe the clinician's message (104 in FIG. 2), translate it into the proxy's language (105), and then re-voice it into spoken audio (106) before it plays on the proxy's device. The proxy thus hears the update in their own language. This happens fairly quickly, thanks to modern AI translation and text-to-speech capabilities, allowing near-real-time multilingual communication.

### Digital Voice Processing

In some embodiments, when messages are sent verbally, they can be digitally processed to remove or reduce indications of illness in the sender's voice. For example, a patient who is very ill might have a weak or raspy voice; the system could process the audio to sound clearer or more energetic (so as not to alarm family with how sick the patient sounds). Similarly, a patient who can't speak could type a text message and the system will convert it to speech using a synthesized voice (potentially even a voice that sounds like the patient's pre-illness voice if such a sample is available). These kinds of voice enhancements ensure that the focus remains on the content of the message rather than any unsettling auditory cues.

### Content Moderation and Stress Analysis

The system can employ AI to ensure that content delivered to the patient is appropriate and not harmful. For example, an analysis of inbound messages (like those from family to patient) can determine if a message might be inappropriate given the patient's condition. This could involve checking the content for highly stressful news or using a voice stress analysis on the patient's responses. If the patient shows signs of distress (through their voice or biometric signals from wearables) after hearing certain messages, the system could intervene. It might, for instance, delay further messages from that sender and notify the proxy that those messages are causing stress. The AI can compare stress indicators before and after a message plays. Metrics like voice stress level, heart rate, or even how forcefully a “stop” button was pressed can be quantified. Based on these, the system can make decisions such as: (a) whether an incoming message's content is appropriate at that moment (if the patient is very fragile, even a normal message could be deemed too exciting, for example), (b) whether the patient is currently in distress and needs human intervention, and (c) whether to play additional messages or hold off if a trend of increasing stress is detected. The overall goal is to ensure communications are helpful, not inadvertently harmful.

### Interactive Content and Media Control

The system supports additional interactive media. For example, white noise or calming sounds can be played on demand. A patient or clinician could say “play white noise” and the device will produce gentle white noise to help mask ambient disturbances. This function can be requested either by the patient (through their device or wearable) or by a clinician remotely (using the clinician's interface to turn on calming audio on the patient's device). Another feature is music therapy integration: patients or caregivers can select music from personal libraries or streaming services to play for the patient. Control of this music can be granted to different parties. For instance, a family member could remotely start a playlist of lullabies at bedtime, or a clinician could stop the music if it's time for a medical procedure. The system can monitor the patient's reactions to the music via AI sensors (as mentioned earlier, detecting facial expressions of pleasure or dislike). If the patient consistently shows a happy response when a particular song plays, the system might play that more often (or conversely skip songs that seem to elicit negative expressions).

### Data Logging and EHR Integration

The system can keep a log of interactions and, importantly, it can share data with electronic health record (EHR) systems if needed. For example, if the app captures that the patient was feeling better at a certain time, or if it records a message from the patient describing their pain level, this information might be flagged as clinically relevant. Certain data from the app (with appropriate consent) can be automatically sent to the patient's EHR as part of their clinical history. For instance, if a patient indicates via the app “I am in pain” and no nurse was present, that could be logged for the doctor to review later. The system can be prescribed by a physician through the EHR as a non-pharmacological intervention (e.g., ordered in the chart that the patient should use this communication device to connect with family as part of their care plan). Hospital pharmacists or other staff might be tasked with setting up the device for the patient upon such an order, treating it somewhat like a prescribed therapeutic device. When the patient is discharged, the device can be unassigned from that patient (wiping any data on it after transferring it to the patient's cloud account). All data (messages, transcripts, etc.) are kept in the patient's account so the patient or their proxy can retrieve them later. In the event of the patient's death, the system may allow the patient's account (and device, if still present) to remain accessible for a time so that grieving family members can continue to send messages (perhaps as a form of bereavement therapy, sending final thoughts “to” the deceased patient's account for their own emotional closure). Those messages might be stored but not played anywhere, or played on a designated memorial device or forwarded to an archive that the family can keep.

In summary, the detailed description above outlines a comprehensive communication system that addresses the unique challenges of connecting patients (especially those in medical or assisted care settings) with their families and healthcare providers. The system leverages modern technologies—from AI-driven translation and sentiment analysis to wearable sensors and encryption—to ensure that communication is seamless, adaptive, and secure. It not only replicates the experience of being there with the patient (through voice, video, and even environmental sharing), but also provides novel features that enhance safety (like automatic fall alerts and stress monitoring) and understanding (like cross-language messaging and explanations of medical terms). By integrating these elements, the invention aims to reduce the isolation of patients, keep families engaged in care, and ultimately improve clinical and emotional outcomes.

### Additional Embodiments and Implementations

The following section is intended to supplement the foregoing description of the invention. Unless otherwise stated, the features, devices, and processes described below may be combined with any of the embodiments set forth in the existing description, or indeed with each other, in any suitable manner. Likewise, references herein to “patients” extend to any user or individual in need of remote or asynchronous communication support, including humans or non-human animals (as discussed further below). Unless the context clearly requires otherwise, statements of functionality should be understood to apply optionally or in various embodiments.

### Sterilizable and Biodegradable Cases

In some embodiments, the patient-facing device (e.g., a tablet, smartphone, or specialized hardware) is physically enclosed in a case designed for easy sterilization and/or built from biodegradable or otherwise environmentally friendly materials. This feature is especially beneficial in high-sterility environments such as intensive care units (ICUs), quarantine areas for highly infectious diseases, or veterinary isolation wards.

### Materials and Construction

The case may be formed of plastics (e.g., polypropylene, polyethylene), silicone, thermoplastic polyurethane (TPU), or treated paper-based composites that exhibit antimicrobial properties or can withstand repeated cleaning with germicidal wipes, alcohol swabs, UV sterilizers, and other standard hospital-grade disinfectants.

In certain embodiments, the case is fully waterproof or water-resistant so it can be rinsed or submerged in cleaning solutions without harming the enclosed device.

For sustainability, the case or shell can be built from biodegradable polymers (for example, polylactic acid (PLA) or polyhydroxyalkanoates (PHAs)) so that when the system is eventually discarded, environmental impact is minimized.

### Sterilization-Compatible Designs

The case can include smooth, rounded edges and a minimal number of seams, thus reducing bacterial harborage sites.

In some embodiments, it includes removable seals or gaskets that can be separately sterilized or replaced.

For hospitals that use UV-based sterilization rooms or cabinets, the material selection may specifically ensure minimal degradation over many UV exposures.

By offering a sterilizable and/or biodegradable case, the invention ensures better infection control and reduced waste, thereby improving suitability for large-scale deployments in clinical or other sensitive environments.

### Integration With Robots or Robotic Arms

In certain implementations, the system can be integrated directly into a movable robot, robotic cart, or robotic arm capable of physically approaching the patient at the patient's request or at scheduled intervals. Such embodiments are beneficial in the following ways:

### Proximity on Demand

If the patient has limited mobility or is bedbound, they may activate a voice command (e.g., “Come here, device”) or press a button on a wearable that causes the robot/arm to drive or pivot into position so the patient can hear or see messages clearly and respond more comfortably.

### Automated Charging and Docking

The mobile robot or robotic arm can return to a charging dock when not in use, ensuring that the patient communication device is always powered.

In some embodiments, the robot can move from room to room, servicing multiple patients in a ward, thereby sharing a single communication “station” among multiple users.

### Touch-Free Interaction and Sterile Transfer

Particularly in infectious-disease units, a robot can help reduce human contact by ferrying the communication device (or built-in screen and speakers) to the patient. This helps protect both the patient and clinicians from cross-contamination.

This approach extends the system's reach to heavily restricted areas (such as quarantine wards or high-containment labs) while preserving the same software-based communication features (asynchronous voice, text, translation, etc.).

### Advanced Imaging and Microexpression Analysis

While the system has generally described AI-based analysis of video for detecting patient mood, distress, or facial expressions, further embodiments incorporate ultra-high-speed and specialized imaging technologies to analyze microexpressions or other subtle physiological indicators:

### High Frame-Rate Cameras and Femto-Photography

In certain implementations, the system may use compressed ultrafast photography (CUP) or femto-photography to capture extremely brief facial microexpressions, subtle pupil dilation, or micro-tremors. Though these cutting-edge methods are still under development, they promise unparalleled temporal resolution.

The system can store or process this high-speed data locally or transmit it (securely) to a server for advanced machine learning interpretation.

### Structured Light and LIDAR

A device's built-in or connected sensor can emit structured light or LIDAR pulses to map the patient's facial geometry in real time and detect micromovements associated with pain, anxiety, or neurological deficits.

Depth maps, in conjunction with normal video frames, enable robust detection of subtle cues like an incipient smile or a wince.

### Thermal and Hyperspectral Cameras

For additional privacy or low-light usage, a thermal camera (such as a FLIR sensor) can detect changes in surface temperature correlated with emotional or physiological states.

Hyperspectral imaging may help track changes in oxygenation or blood flow near the skin in real time.

### Use of AI Models on Microexpressions

The system may employ advanced neural networks or specialized microexpression classifiers (based on theories by, e.g., Paul Ekman) to interpret fleeting facial expressions (under 0.5 seconds) as indicators of distress, confusion, or other emotional states.

The resulting data can be used to adapt communication strategy, e.g., pausing certain messages if they appear to cause agitation.

These advanced sensor modalities remain fully optional within the invention's scope but illustrate how the communication system can be enhanced by next-generation imaging technologies.

### Extended Cryptographic Implementations

As described in the main specification, the invention supports robust encryption for HIPAA-compliant data protection. Certain embodiments expand these security mechanisms in the following ways.

### Consumable One-Time Keys (OTK) or “Disposable Keys”:

Each transmitted message can be encrypted with a one-time symmetric key that is valid for a single message exchange only.

After playback or a short expiration window, that key is discarded. A fresh key is generated for the next message, making long-term key compromise more difficult.

### Hardware Security Modules (HSM) and Trusted Platform Modules (TPM):

The system's keys or cryptographic materials are kept in dedicated hardware enclaves. Even if the main operating system is compromised, attackers cannot extract encryption keys.

### Multi-Factor Authentication (MFA) for Sensitive Actions

In especially high-security environments, certain message types (e.g., urgent or private doctor-to-proxy communications) require the user on the receiving end to supply two or more authentication factors (e.g., password+biometric or password+time-based one-time passcode) before decrypting the content.

### Encrypted Peer-to-Peer Failover (See Section 7 Below)

In the event that the central server is unreachable, devices are capable of initiating direct peer-to-peer (P2P) encrypted channels, bypassing the normal cloud routing but retaining end-to-end encryption.

By expanding cryptographic rigor, these embodiments make the system suitable for high-stakes or highly regulated environments where data leakage is particularly hazardous.

### Use With Non-Human Animals

While the main embodiments focus on human patients, the underlying technology can also facilitate communication with or about non-human animals:

### Veterinary Applications

In veterinary ICU or quarantine settings (e.g., exotic animal hospitals or high-biosecurity farms), staff may need an asynchronous method for relaying status updates to an owner who cannot physically visit.

The device can capture audio, video, or sensor data from an animal's enclosure—e.g., movement, vital signs if wearable collars or harnesses are used—and transmit it to the remote caretaker.

### Behavioral Cues and Interaction:

For some companion animals (dogs, cats, horses), remote owners can send reassuring voice messages (e.g., the owner's own recorded voice or a re-voiced message) that automatically play for the animal in the enclosure.

AI analysis of the animal's posture or vocalizations (barking, meowing, etc.) may inform a caretaker if the animal is distressed, depressed, or restless.

### Translational Models (Human<->Animal Sound Cues):

Although “animal language” translation is not as developed, certain AI classifiers can interpret approximate emotional states in animal vocalizations. This data is then provided to the remote owner or vet staff, akin to an emotional tagging system for humans.

In short, even though animals cannot typically operate the device themselves, the system's asynchronous messaging and advanced sensor analysis can bridge the gap between the non-verbal animal “patient” and remote caretakers or veterinarians.

### AI-Based Scam Detection and Elder-Abuse Screening

In some implementations, the system includes content moderation or scam detection features designed to protect vulnerable individuals (e.g., elderly patients with cognitive impairment) from malicious or manipulative messages:

### Inbound Message Screening

Each newly arrived audio or text message is analyzed by a specialized AI module for indicators of fraud, exploitation, or emotionally abusive language.

Examples include detecting pressure to transfer money, requests to reveal personal information (e.g., Social Security numbers), or strong intimidation tactics.

### Elder-Abuse Warnings

If the system detects patterns consistent with emotional or financial abuse—such as repeated harassing language, extreme guilt-tripping, or suspicious financial solicitations—it can flag these messages for manual review by a trusted proxy or facility administrator.

In severe cases, the system may temporarily block further messages from the suspected sender until a human gatekeeper intervenes.

### Adaptive Machine Learning

The scam detection may rely on natural language processing (NLP) techniques and user feedback. Over time, as flagged messages are confirmed or dismissed, the model becomes more accurate at identifying manipulative or harmful content.

### Beneficial Lies and Tone Adjustment

Conversely, if a caregiver or family member chooses to soften certain messages to avoid stressing the patient (e.g., withholding bad news from a delirious or palliative patient), the system can optionally provide an “AI rewriting” function to deliver a gentler version of the message. This is the ethical flip side of preventing elder abuse: the system can also help reduce distress by rephrasing messages in a calmer, more reassuring tone, subject to user control.

Such safeguards further the invention's core mission of protecting isolated or vulnerable individuals.

### Peer-to-Peer Fallback Communication

Although the system is described primarily as server- or cloud-based for message storage and routing, certain embodiments enable direct peer-to-peer (P2P) encrypted communication when the server is unreachable or when privacy rules forbid external cloud routing:

### Automatic Failover

If the communication processing component (e.g., the hosted server) becomes unresponsive, the patient device and a remote family device can attempt a local network or Wi-Fi Direct connection, transferring voice or text messages using ephemeral encryption keys.

This ensures that sudden server downtimes do not isolate the patient from essential communication.

### Local Network-Only Mode

In especially strict settings (such as certain penal institutions, secure mental health wards, or remote field hospitals with no reliable internet), the system can operate entirely “offline” by discovering devices on the same local network or via Bluetooth mesh.

All messages remain encrypted end-to-end and never traverse an external server.

### Notification of P2P Status

The system may display to both sender and recipient that a P2P fallback is active, so they understand potential limitations (e.g., no cloud archiving or AI translation unless locally available).

In these embodiments, all the same encryption and transcription features can still function if the patient device or local node has those modules installed and has enough processing capability. Once connectivity to the main server is restored, the system can optionally sync the messages for archival or re-check with advanced AI modules.

### Enhanced Channel Management

The core specification discloses multi-user channels that may be created, joined, or closed as needed. Certain implementations add more granular channel membership controls and “private vs. shared” tagging:

### Patient Removal of Members

Beyond the general notion of inviting or removing members, some embodiments explicitly allow the patient (or proxy) to remove any participant from the channel (except perhaps the mandatory proxy). The user interface for this feature can be simplified to a “remove user” icon next to each participant's name.

Private vs. Shared Tags

When family members tag a message (e.g., “funny,” “urgent,” or “confusing”), they can mark the tag as “private” so it is only visible in their personal interface (useful if a family member wants to keep private notes for reference). Alternatively, “shared” tags are visible to all participants.

### Tag-Based Permissions

In advanced configurations, certain tags (like “clinical data” or “serious request”) might only be viewable by the clinician or proxy, thus ensuring sensitive content is restricted. For instance, if a relative's message is labeled “financial assistance needed,” the system might make that message accessible only to the proxy rather than the entire extended family group.

These optional features allow for more nuanced management of multi-user conversations, consistent with maintaining patient autonomy and respecting user privacy preferences.

### Memory/Event Supplementation and Automated Summaries

While the main text describes how the device might continuously or periodically record environmental data for memory-impaired patients, some embodiments expand or refine these features:

### Continuous or Triggered Recording

The system can store short rolling video or audio buffers (e.g., the last 10 minutes) and keep only the segments during which significant events (patient movement, visitors entering, alarms sounding) are detected.

This helps reduce the storage burden while still capturing potentially important episodes.

### AI Summaries

The system can automatically generate a textual summary of events for the patient or proxy, such as “You had three visitors today between 1 PM and 3 PM. At 2:15 PM you had a phone call from your daughter; at 2:35 PM you napped for 30 minutes.”

In patients with memory deficits, these summaries can be played back on demand or regularly scheduled for reorientation: “Today is Tuesday, you had breakfast at 8:30 AM, and a nurse visited at 9:00 AM.”

### Privacy Filters

The system can “blur” or strip identifying details before storing or transmitting event logs. For instance, it could record that a visitor came in without retaining the visitor's face or name. This is particularly useful where privacy laws or the patient's preferences dictate minimal data retention about third parties.

### Additionally Embodiments

Case to secure the device can be sterilized by using a material that can be wiped with a cleaning solution, such as alcohol or germicide, applied with a swab or cloth on material that will not get damaged from the cleaning solution. Cases can also be sterilized using UV light of the type used to sterilize a room or in a container used to sterilize an instrument. The case is built from biodegradable materials.

Casa can be integrated into a moving robot or robotic arm to approach the patient at their request and provide for their communication needs.

Automatically change device functionality based on data from other sensors, such as specific oxygen (SpO₂), blood pressure, heart rhythm monitors. For example, when the patient is sleeping the devices pauses automatic playback and waits to play the messages in their original incoming order when the patient is awake.

The device may be worn on the wrist with applications on the device and sensors. The app running on the wrist device can transmit data from the wrist device to the app on the mobile device, which may change its behavior or may send a notification to the staff or the family channel, depending on the sensor reading and the configuration.

The app takes input from AI-enabled IoT devices that monitor the patient's face and/or movement. When an adverse event, such as fall is detected, the app can make an automated phone call to the nursing station and/or the designated health care proxy

Uses hospital device measurements to determine whether the patient is to not be disturbed so if the readings are consistent with sleep, or the patient is on lots of opiates, it would delay any messages to avoid disturbing the patient.

Actuation of the app can be triggered by speaking a codeword for patients who are unable to press a record button, such as “Hey VoiceLove, send a message to the family channel saying XYZ.” This task may be accomplished either as a single request or as a dialog where the app makes inquiries for missing information, such as the channel or the message.

Voice to text combined with functions, such as one when saying “help” first pages the nurse's app and if not deactivated within a configurable period, an alert message is sent to the family.

Ability to record audio and optionally video messages to be sent to a communication channel and automatically played on the destination devices that are part of the channel unless the destination device is paused.

The app includes a pause feature whereby the app may be placed in a pause mode for a specified period, pausing automatic playback. Pause may be scheduled as a recurring pause period, such as each night. The sender of a message sees an indicator that the receiving device is in pause mode and when the pause mode is scheduled to end.

A set of user accounts is connected to a channel. Any user can create a channel and invite others via email or text message to join the channel. A user may participate in many channels. For patients and residents of long-term care facilities, the initial channel is set up by the clinical staff to ensure an initial private connection is made to an authorized family member.

When a channel is being shut down, a family member may elect to download the complete history of voice messages for long-term storage, safe keeping, and later playback. All message history for each channel that is being shut down is kept for a few weeks to allow time for the proxy to agree to safekeep the voice messages.

When used in a facility setting, the facility administrative console can schedule a pause of all devices in the facility, such as during the night. A short message can be played to announce the pause.

When used in a facility setting, the facility administrative console can broadcast a message to all devices in the facility or to a specific subset of devices, such as devices in a particular unit, department, floor, building or wing.

A facility administrator can broadcast a message for immediate playback to all devices in their facility or to specific units, such as all devices in the adult ICU units.

Voice messages are automatically transcribed, and the transcriptions can be automatically translated into the language of the receiver's choice then turned into a spoken voice message in that second language and played automatically.

Artificial Intelligence (AI) is used to find key terms and important concepts in the transcriptions so that the AI can apply keyword tags to the voice messages. Tags can include concepts, tone, agitation, or other emotional insights.

Filters can be applied to a set of voice messages to select by source, target audience, date, date range, time of day, range of times, and keyword tags.

Voice message history can be sorted by source, target audience, date/time, and keyword tags, as well as emotional components such as message sentiment and intention.

Receivers can add tags to voice messages that can be held private and only made available to them or that they can share with all members of the communication channel of the voice message.

When a receiver gets a voice message from a clinician that includes medical terms, they can ask for the terms to be explained, including the ability to explain the terms in a second language.

Since some patients and residents may have trouble remembering events from day to day, a message can be tagged as a reorientation message to be played with a recurring frequency, such as daily or hourly.

Automatic playback may be paused by the clinician for a specific period or by a schedule, such as during sleep, a procedure, or some other activity where the message playback would be disruptive.

Channel members can see when the intended recipient's app is paused, such as for healthcare activities or nighttime.

All devices in a facility can have their automatic playback controlled at the facility level by the administrator, such as during a lights-out period at night.

Facilities can set a schedule of “virtual visiting hours” when voice messages can be played automatically.

Senders of messages will be informed of virtual visiting hours.

Ability for family to select from a set of messages either prerecorded or that they record for playback on the patient's device, including pleasant nature sounds, human heartbeat, nursery rhymes or songs, encouraging messages, such as from famous people or special friends, or songs or messages recorded by the family member that the family member wants to have played on a recurring basis, such as daily or hourly.

Ability for the patient to remove people from a channel, except their proxy.

A user can request verbally that the app play a set of messages, such as play unheard messages from a specific person that were sent over the last week.

White noise play functionality is integrated an can be requested by the patient or the clinician using the patient's phone in clinician-mode.

Music selection from personal libraries or purchased from music vendors to play to the patient is enabled and can be controlled by the patient, the clinician, or a family member.

Music playing or pausing monitored by AI-enabled sensors to detect facial expressions confirming like or dislike.

Data from the app that are determined to have clinical relevance are shared with the EHR for clinical history purposes.

Device with communication software can be prescribed from the EHR by a physician as non-pharma family intervention.

Device can be dispensed or set up by pharmacists or other clinical or administrative workers in the ICU.

Device is assigned to a specific patient and unassigned when the patient is discharged. All data belonging to the patient is kept within the VoiceLove patient account and available to the patient or health care proxy for later download.

In the event of patient death, device and communication software assigned to the patient can be used by grieving remote family members that seek closure by sending messages to the account of the deceased.

A pleasing alert with a short delay is played prior to automatic message playback in case someone in the room prefers to pause the messages due to care activities, visitors, privacy, concerns, etc.

Clinician's desktop dashboard app can connect to a patient's proxy to allow the clinician to gather their thoughts at their workstations before recording a message for the patient's proxy.

Data from EHR required for device/software optimal performance are shared from the EHR

App sends a pleasing alert with a short delay prior to automatic message playback in case someone in the room prefers to pause the messages due to care activities, visitors, privacy, concerns, etc.

Add voice stress analysis, quantitative data about the button press, and voice to text AI analysis to determine

Whether content of an inbound message is appropriate based on message content or patient stress level.

Whether the patient is in distress.

Compare the stress level before and after listening to a message. Based on voice stress, use that data to inform decisions about playing back additional messages from the sender.

The device can be connected to a projector that can project photos onto the ceiling or wall, or stream the sender's video.

Automatic translation of message transcriptions into a second language selected by the recipient and voicing of these transcribed and translated messages.

A communication system for remote virtual visitation, comprising at least one patient-side communication device and at least one remote user communication device in network communication with each other, wherein the system is configured to record an audio message from a first user on a first device and transmit the audio message to a second device associated with a second user, and further configured to automatically play the audio message on the second device upon receipt, unless playback on the second device is intentionally paused, thereby enabling asynchronous voice messaging between the first user and the second user without requiring the users to interact in real time.

The system of claim 1, wherein each recorded audio message is automatically converted to text to produce a transcript, and the system stores the transcript in a searchable archive such that authorized users can retrieve and review message content by searching for keywords or other criteria.

The system of claim 1, wherein the system further comprises a translation module configured to translate the audio message from an original language of the first user into a different language of the second user, and a revoicing module configured to generate a synthesized audio rendition of the translated message in a voice that mimics the voice of the first user, such that the second user hears the message in the second user's preferred language while preserving characteristics of the first user's voice.

The system of claim 1, wherein the audio messages are not limited to voice alone and can include or be accompanied by additional media elements selected from the group consisting of: video clips, text, images or slideshows, music or songs from a playlist, virtual or augmented reality content, and tactile or olfactory cues, and the system is configured to deliver said media elements to the second device in synchronization with or in addition to the audio playback.

The system of claim 1, wherein communications through the system are secured with end-to-end encryption such that each audio message is encrypted on the sending device and decrypted only on the intended receiving device, thereby protecting the confidentiality of the messages and complying with privacy requirements.

The system of claim 1, wherein the system further comprises an artificial intelligence module that analyzes the content or characteristics of the audio messages and detects emotional or contextual cues, and in response provides a recommended next action or communication adjustment to a caregiver or moderator user (via a user interface notification) to improve or tailor subsequent messages for the benefit of the patient.

A method of facilitating remote visitation and communication for a patient using an asynchronous messaging platform, comprising: (a) recording an audio message from a sender via a first user device associated with the sender; (b) transmitting the audio message over a network to a second user device associated with the patient; and (c) automatically playing back the audio message on the second user device upon receipt, whereby the patient hears the sender's voice message without needing to actively initiate or answer a call in real time.

The method of claim 7, wherein the step of recording the audio message includes monitoring the audio input for a period of silence and automatically terminating the recording when no voice input is detected for a predetermined duration, thereby delimiting the message end without requiring the sender to manually stop the recording.

The method of claim 7, further comprising converting the recorded audio message into a text transcript using speech-to-text processing and storing the transcript in a computer-readable storage medium associated with a message archive for the patient.

The method of claim 7, further comprising translating the content of the audio message from a first language into a second language and synthesizing a translated audio message in the second language, and delivering the translated audio message to the second user device such that the patient hears the message in the second language.

The method of claim 7, further comprising providing a search functionality by which an authorized user can query stored transcripts of past audio messages based on one or more criteria selected from: a specific word or phrase contained in the message, the identity of the sender or recipient of the message, a tag or label associated with the message, the sentiment or emotional tone of the message, and a date or time range during which the message was sent.

The method of claim 7, wherein the second user device automatically plays incoming audio messages in real time as they arrive unless the device is in a paused or do-not-disturb mode, and if the device is in such a mode, the method includes queuing the received audio messages and then automatically playing the queued messages in their original order of receipt once the device is unpaused.

A protective case for a mobile communication device, comprising a housing adapted to encase and protect the mobile communication device, wherein the housing is constructed of a material that remains unharmed by sterilization procedures, including exposure to disinfectant cleaning solutions and ultraviolet light, such that the case with the enclosed device can be repeatedly sanitized for use in healthcare or other sterile environments without damage.

The protective case of claim 13, wherein the housing is configured to withstand sterilization by ultraviolet-C (UV-C) light exposure, such that the material does not substantially degrade, warp, or lose functionality after repeated UV-C sterilization cycles.

The protective case of claim 13, wherein the housing is configured to withstand cleaning with chemical disinfectants, including alcohol-based solutions and germicidal wipes, without deterioration, discoloration, or loss of structural integrity.

The protective case of claim 13, wherein the housing further comprises one or more attachment features selected from the group consisting of: adjustable straps, hook-and-loop fasteners, buckles, clamps, or hanging loops, thereby enabling the case to be secured to a patient's bed, chair, clothing, or surrounding fixtures to position the mobile communication device for hands-free use by the patient.

The protective case of claim 13, wherein the housing is at least partially composed of a biodegradable material that allows for environmentally friendly disposal of the case after its useful life, while still maintaining required durability and sterilization resistance during use.

The protective case of claim 13, wherein the housing includes a transparent window or membrane aligned with the screen of the mobile communication device and configured to allow touch input and viewing of the device's display, so that the device can be operated and monitored while remaining enclosed in the sterilizable case.

A robotic communication system for assisting patient communications, comprising: a mobile robot or robotic arm and a patient communication device mounted on the robot or robotic arm, wherein the robot or robotic arm is configured to automatically move the communication device into proximity with the patient in response to a summon command or according to a predetermined schedule, thereby allowing the patient to access and interact with the communication device without the patient needing to physically retrieve or hold the device.

The system of claim 19, wherein the mobile robot or robotic arm is configured to be summoned by the patient via a voice command or a wireless call signal, such that the patient can use a spoken phrase or button press to cause the robot to approach the patient's location with the communication device.

The system of claim 19, wherein the robot comprises a docking station or charging base and is further configured to autonomously return to the docking station when not in active use, the docking station providing power to recharge the robot and the mounted communication device.

The system of claim 19, wherein the robot or robotic arm adjusts the position and orientation of the communication device by moving in at least one degree of freedom selected from vertical height adjustment, distance from the patient, and angular orientation, thereby positioning the device's screen, camera, and speakers optimally relative to the patient for effective communication.

The system of claim 19, wherein the mobile robot is equipped with navigation sensors and programmed to autonomously navigate within a facility to locate the patient, and to avoid obstacles or hazards while transporting the communication device to and from the patient.

The system of claim 19, wherein the communication device mounted on the robot is enclosed within a protective case comprising a sterilizable material, such that the device remains protected and germ-free while being delivered to or moved near the patient by the robot.

A communication system adaptive to a patient's condition, comprising a patient communication device equipped with one or more sensors or inputs that monitor the patient's state, wherein the system is configured to automatically modify the delivery of voice messages based on the patient's condition as determined from the sensor data, including pausing or delaying message playback upon detecting that the patient is asleep, sedated, or otherwise not in a suitable state to receive messages, and resuming playback when the patient becomes receptive.

The system of claim 25, wherein the one or more sensors include vital sign monitors measuring at least one of: heart rate, respiratory rate, blood pressure, and oxygen saturation, and the system is configured to interpret vital sign patterns indicative of sleep or low consciousness and to pause automatic message playback during such periods.

The system of claim 25, wherein the system resumes normal message playback (playing any queued messages) once the sensor data indicates that the patient has awoken or returned to an alert state, so that the patient can hear any messages that arrived while playback was paused.

The system of claim 25, wherein if message playback is paused due to the patient's state, incoming messages are retained in a queue and, upon the patient becoming receptive, the system plays the queued messages in the order they were received, thereby preserving the original sequence of communications.

The system of claim 25, wherein the one or more sensors comprise an ambient environmental sensor selected from: an ambient light sensor, a sound level sensor, or a room motion sensor, and the system is configured to infer from environmental conditions (including detecting darkness or quiet consistent with nighttime or the patient resting) that message playback should be delayed or muted until conditions indicate the patient is likely awake or available.

The system of claim 25, wherein the patient communication device is operatively connected to external patient monitoring equipment or electronic medical record inputs that provide data about the patient's status, and the system is configured to use said external data-including indications of the patient being under sedation, on certain medications (e.g. high doses of opioids), or undergoing a medical procedure—to determine that the patient should not be disturbed, and accordingly to postpone or suppress message playback until the external data indicates it is appropriate.

A communication system with wearable sensor integration, comprising: a primary communication device associated with a patient and at least one wearable device worn by the patient, the wearable device including one or more sensors for monitoring a physiological or movement parameter of the patient and a wireless transmitter, wherein the wearable device is configured to transmit sensor data to the primary communication device, and the primary communication device is configured to adjust its operation or send an alert based on the sensor data according to a predetermined rule set.

The system of claim 31, wherein the wearable device is a smart wristband or smartwatch that measures the patient's vital signs and/or movements, and the one or more sensors on the wearable device include at least one of: a heart rate sensor, an accelerometer, a blood oxygen sensor, or a blood pressure sensor.

The system of claim 31, wherein upon the wearable device detecting an abnormal reading or event in the patient's parameters (selected from a group consisting of: heart rate outside a target range, a fall or sudden movement indicative of an accident, or prolonged inactivity beyond a threshold), the primary communication device automatically sends an electronic alert or notification to medical staff and/or to a designated family member through the communication system.

The system of claim 31, wherein the primary communication device dynamically modifies how it plays or records messages in response to data from the wearable device, including adjusting the volume or frequency of message playback if the patient's physiological readings (from the wearable) indicate stress or agitation, or conversely delaying non-urgent communications if readings indicate the patient is resting or in a fragile state.

The system of claim 31, wherein the wearable device communicates with the primary communication device via a short-range wireless protocol, and the system ensures reliable data transmission of the sensor readings to the primary device in real time or near real time (for example, via Bluetooth Low Energy or a similar wireless link).

The system of claim 31, wherein a plurality of wearable devices or sensors on the patient are integrated through the primary communication device, such that data from multiple sources (including, for example, a wearable ECG monitor, a fall-detection pendant, and a glucose sensor) can be aggregated and used by the communication system to make comprehensive decisions about alerting caregivers or modifying communication delivery to the patient.

A patient monitoring communication system for detecting patient emergencies and notifying caregivers, comprising: a patient communication application running on a device and one or more external sensors or IoT devices monitoring the patient's environment or condition, wherein the communication application is configured to receive input from the one or more sensors and, upon detecting an adverse event involving the patient, automatically initiate an alert communication to medical staff or a designated caregiver without requiring the patient to manually send a message.

The system of claim 37, wherein the one or more external sensors include a camera with an AI-based video analysis module that is capable of recognizing when the patient has fallen or is in an abnormal position indicating a possible fall, and when such an event is detected the communication application automatically generates an alert.

The system of claim 37, wherein the alert communication initiated by the application comprises at least one of: an automated phone call, an emergency text message, or an in-app notification sent to a nurses’ station or on-call medical personnel, and optionally a simultaneous alert to the patient's designated health care proxy or family member, informing them of the detected adverse event.

The system of claim 37, wherein the monitoring includes analyzing the patient's facial expressions, voice, or movements via the sensors to determine if the patient is in distress or attempting to seek help (for example, repeatedly saying a particular phrase or making a hand gesture), and the system treats such indications as triggers to send an alert or to request a check-in by caregivers.

The system of claim 37, wherein the application is configured such that, in response to detecting an adverse event, it first sends an alert to on-site medical staff and, if no acknowledgment or response is received within a predefined time interval, the system escalates by sending a follow-up alert or call to the patient's designated family member or proxy.

The system of claim 37, wherein the one or more external sensors comprise a wearable fall-detection sensor or motion detector on the patient that communicates with the communication application, and upon detection of a sudden acceleration or impact characteristic of a fall by the wearable sensor, the application triggers the automatic alert to caregivers.

A voice-activated communication method for hands-free messaging by a patient, comprising: continuously monitoring audio input from the patient for a predefined voice command that serves as a trigger, and when the predefined voice command is detected, automatically initiating a message recording process on a communication device, then capturing the patient's spoken message, and sending the spoken message to a predetermined recipient or communication channel without the patient having to manually operate the device.

The method of claim 43, wherein the predefined voice command is a unique wake-word or phrase (optionally incorporating a system name) that causes the system to start recording a voice message when spoken by the patient, thereby enabling the patient to initiate message sending by voice alone.

The method of claim 43, wherein after initiating the message recording process, the system engages in an interactive voice dialogue with the patient to obtain additional information needed to send the message, by prompting the patient for details such as the intended recipient or channel and confirming the message content if the initial voice command did not specify all necessary parameters.

The method of claim 43, wherein the predefined voice command includes an indication of the message recipient and content in a single utterance (as an example, “Hey [App Name], send a message to my family that I'm okay”), such that the system can parse the command, determine the target recipient group and the message body, and send that message with no further input.

The method of claim 43, wherein the system provides feedback to the patient during the voice-activated message sending process, including audibly confirming that the message was recorded and is being sent, or playing back the recorded message for patient approval before transmission, to ensure accuracy and give the patient confidence in the hands-free operation.

The method of claim 43, wherein the voice-activation feature is configurable and designed for patients with limited mobility or dexterity, such that it can be enabled for those patients who cannot press physical controls, and disabled or adjusted (for example requiring a different code phrase) in environments where accidental activation by ambient speech is a concern.

A communication method for cross-linguistic messaging, comprising: receiving an audio message spoken in a first language from a sender; generating a text transcript of the audio message in the first language; translating the transcript from the first language into a second language to produce a translated text; synthesizing a second audio message by converting the translated text into speech in the second language with a voice that resembles the sender's voice; and delivering the synthesized second audio message to a recipient who understands the second language.

The method of claim 49, wherein synthesizing the second audio message includes preserving vocal characteristics and emotional tone of the sender, such that the translated speech maintains the sender's unique voice qualities, intonation patterns, and emphasis, thereby making the translated message sound as if spoken by the sender in the second language.

The method of claim 49, wherein translating the transcript further includes interpreting cultural references, idiomatic expressions, or context-specific terminology from the first language and converting them into equivalent expressions in the second language that convey substantially the same meaning and emotional nuance to the recipient.

The method of claim 49, wherein the method further comprises generating a visual output to accompany the translated audio message when appropriate, including at least one of: an avatar or animation depicting sign language or gestures corresponding to the message content, or on-screen text subtitles in the second language, in order to convey emphasis or context that might otherwise be lost in audio-only translation.

The method of claim 49, wherein if the recipient's device or communication preferences indicate that the original language is acceptable or preferred, the method bypasses the translation and revoicing steps and delivers the original audio message for immediate playback, thereby avoiding any delay introduced by translation when it is not needed.

The method of claim 49, wherein the translation and revoicing of the message are performed in near real-time such that the time elapsed between the sender completing the audio message in the first language and the recipient beginning to hear the corresponding message in the second language is minimized, enabling fluid cross-lingual conversations.

A communication platform with multi-user channels, comprising a server or cloud-based service and associated software applications, wherein the platform maintains a plurality of communication channels, each channel being associated with a particular patient or group and linked to a set of user accounts, and the platform is configured to permit an authorized user to create a new channel for a patient, invite other users to join the channel via electronic invitations, and enable all users in the channel to exchange voice and other media messages with each other securely.

The platform of claim 55, wherein for a patient in a healthcare facility, the initial communication channel for that patient is established by clinical staff or administrators, and at least one family member or proxy of the patient is invited as an initial member of the channel after verification, so as to ensure that the channel begins with an authorized participant who can then invite additional family or friends.

The platform of claim 55, wherein individual users can belong to multiple channels simultaneously (for example, a user might be a family member in several patient channels and also a patient in their own channel), and the platform provides an interface for the user to switch between channels and manage their participation (including joining or leaving channels and adjusting notification preferences for each channel).

The platform of claim 55, wherein invitations for a new user to join a channel are sent via electronic message (including email or SMS) containing a secure link or code that, when used by the new user, allows them to create an account or log in and be added to the specific channel, thereby streamlining the onboarding of invited participants.

The platform of claim 55, wherein each communication channel maintains a history log of messages exchanged (including audio message files, transcripts, and other media) and the platform enables authorized members of the channel to download or export the complete message history for long-term storage or safekeeping, particularly when a channel is to be closed or the patient is discharged.

The platform of claim 55, wherein access to the channels and messages is controlled by authentication and role-based permissions, and all communications within a channel are encrypted, such that only channel members with proper credentials can access the message content, thereby ensuring privacy and regulatory compliance for sensitive communications (including compliance with healthcare information privacy laws).

A centralized communication management system for facilities with multiple patient communication devices, comprising an administrative console application in communication with a plurality of patient communication devices, wherein the administrative console allows an authorized staff user to remotely control certain functionalities of the patient devices including broadcasting messages to multiple devices simultaneously and placing one or more of the devices into a pause mode or resume mode for message playback.

The system of claim 61, wherein the administrative console is configured to schedule or initiate a facility-wide (or group-specific) quiet period by sending a command to pause automatic message playback on selected patient devices for a defined duration, and optionally to broadcast a short notification message to those devices announcing the start or end of the quiet period (for example, an announcement at night that devices will be in “do not disturb” mode until morning).

The system of claim 61, wherein the administrative console provides a broadcast function enabling an administrator to select a target set of patient communication devices (ranging from all devices in the facility to those in a particular unit, floor, or group) and send an audio or audio-visual message that will play on all selected devices, thereby disseminating information or emergency announcements to multiple patients simultaneously.

The system of claim 61, wherein when a communication channel or patient device is to be deactivated (such as when a patient is discharged or a communication service is being discontinued), the administrative console facilitates the preservation of message data by allowing staff or the patient's proxy to initiate a download or transfer of the complete voice message history from the device or channel to a secure storage location before shutdown.

The system of claim 61, wherein the administrative console includes an option to mark a broadcast message as urgent, such that an urgent broadcast will override any active pause or do-not-disturb setting on the target devices and play immediately (for example, in the case of a safety alert or evacuation notice), whereas non-urgent broadcasts respect the pause settings and queue until the pause is lifted.

The system of claim 61, wherein the system implements hierarchical access controls for different administrative roles, so that only users with a high-level role (e.g. facility administrator) can broadcast to all devices facility-wide, while users with more limited roles (e.g. unit manager) can broadcast or pause devices only within their assigned unit or group, thereby preventing unauthorized or inappropriate use of mass communication features.

A method of augmenting patient memory and awareness using a communication device, comprising: continuously or periodically capturing audio and/or video data of events around a patient via one or more sensors on the patient's communication device or connected to it; analyzing the captured data to detect and identify notable events or interactions involving the patient; generating a summary record of the events in a human-readable form; and presenting the summary to the patient or authorized personnel to aid in recalling or reviewing the events.

The method of claim 67, wherein analyzing the captured data includes using computer vision techniques to recognize and log occurrences of visitors or staff in the patient's room and detect significant patient activities or movements, and the summary record includes entries with timestamps and descriptions such as identifying a person (by name or label) who visited and the time and duration of the visit, or describing key actions (e.g. “patient stood up and walked 5 steps at 3:00 PM”).

The method of claim 67, wherein the method further comprises applying privacy filters to the captured data such that personal identifying details of individuals (other than the patient) are obscured or anonymized in the stored data and summary, including techniques like blurring faces in video, anonymizing names, or describing individuals generically (for example, “a nurse” instead of the nurse's name), to protect privacy while still conveying the occurrence of the event.

The method of claim 67, wherein the audio and/or video data captured is stored locally on the communication device or on a secure server for a limited retention period, and is accessible to medical staff for review as needed (for instance, to determine the sequence of events leading to a fall or medical incident), after which the data is automatically deleted or overwritten unless flagged for preservation.

The method of claim 67, wherein the summary of events includes chronological annotations that can be output as text or synthesized speech, giving the patient an overview such as, “Today, your daughter Jane visited from 1:30 PM to 2:00 PM. You had a physical therapy session at 4:00 PM. At 5:30 PM, nurses changed your bandages,” thereby helping orient or reassure patients who have memory gaps or confusion about their day.

The method of claim 67, wherein the communication device is operatively connected to additional sensors selected from the group consisting of: LiDAR depth sensors, infrared or thermal imaging cameras, and other environmental or biometric sensors, and data from these sensors is incorporated into the analysis to capture events (such as movement during low light conditions or changes in room temperature) and to improve the accuracy and richness of the event summary provided to the patient or caregivers.

### SUMMARY

All of the foregoing embodiments and features-ranging from sterilizable/biodegradable cases, robotic integration, specialized ultra-high-speed cameras for microexpression analysis, advanced cryptographic detail, use with non-human animals, scam/abuse screening, peer-to-peer fallback, nuanced channel membership control, and expanded memory supplementation—can be incorporated singly or in any combination with the core system described in the main specification. These enhancements further illustrate the breadth of possible implementations, ensuring that the invention can be adapted to a wide variety of clinical, homecare, veterinary, or other specialized scenarios.

Unless otherwise stated, these features are not mutually exclusive: one embodiment may implement multiple features simultaneously while another embodiment implements only a subset, all while remaining within the scope of the invention.

1. A communication system adaptive to a patient's condition, comprising:
a patient communication device with a display configured to present a set of selectable symbols representing patient needs or messages, and at least one input sensor configured to detect non-verbal input from the patient to select one of the symbols;
at least one caregiver communication device configured to receive a selected message from the patient communication device and to display or output the message to a caregiver;
a control unit comprising at least one processor and memory, operatively connected to the patient communication device and the caregiver communication device, the control unit being programmed to interpret the patient's non-verbal input as a selected message, transmit the message to the caregiver communication device, and subsequently receive an acknowledgment or response from the caregiver communication device;
wherein the system is adaptive to the patient's condition such that the control unit adjusts one or more communication parameters based on patient-specific data, including automatically calibrating input detection sensitivity or symbol display characteristics according to the patient's physical or cognitive abilities, and adjusting the content or format of the symbols in response to changes in the patient's condition.

2. The communication system of claim 1, wherein the input sensor comprises an eye-tracking interface configured to monitor the patient's gaze on the display, and the control unit determines the selected symbol based on a detected focus or dwell of the patient's gaze on that symbol.

3. The communication system of claim 1, wherein the control unit is further configured to analyze the patient's interaction with the patient communication device to assess a mental or emotional state of the patient, by monitoring metrics selected from the group consisting of: gaze stability, reaction time to select symbols, accuracy of symbol selection, blink rate, and interaction frequency; and wherein the system generates an alert to the caregiver communication device if the analysis of said metrics indicates a deviation associated with a change in the patient's mental state (such as onset of delirium, fatigue, or distress).

4. The communication system of claim 1, wherein the control unit is configured to detect or predict a health event in the patient by evaluating patient-specific data collected during use, the data including at least one of: the patient's input interaction patterns and physiological sensor data; and wherein upon detecting a threshold condition indicative of a potential adverse health event, the control unit automatically transmits an alert or message to the caregiver communication device to prompt immediate caregiver attention.

5. The communication system of claim 1, wherein each symbol displayed on the patient communication device is represented as an icon or graphical indicium along with an optional text label, and wherein the symbols include icons for basic needs, pain levels, and emotional states.

6. The communication system of claim 5, wherein at least one symbol, when selected by the patient, causes the patient communication device to display a hierarchical submenu of secondary symbols that provide more specific options related to that symbol (such that selection of a general category leads to a second set of more detailed symbols), and the control unit interprets a selection from the submenu as a more specific message before transmitting it to the caregiver communication device.

7. A communication system, comprising a patient communication device and a caregiver communication device as in claim 1, further including wearable sensor integration, wherein:
the system includes one or more wearable sensors configured to be worn by or attached to the patient, the wearable sensors being capable of collecting physiological data from the patient and communicating said data to the control unit;
the control unit is programmed to incorporate the physiological data from the wearable sensors into the adaptive operation of the communication system, by monitoring the patient's physiological state and modifying or prioritizing communications based on that state;
wherein the wearable sensor data includes at least one of: heart rate, blood oxygen level, blood pressure, respiratory rate, sleep pattern, or physical activity, and the control unit uses this data to detect changes in the patient's condition or urgency of needs.

8. The communication system of claim 7, wherein the one or more wearable sensors comprise a heart rate monitor, and the control unit is configured to receive heart rate data in real time and trigger an alert or modify the patient's available communication options if the heart rate exceeds a predetermined threshold or pattern (indicative of pain, stress, or a medical event).

9. The communication system of claim 7, wherein the one or more wearable sensors comprise a sleep or activity tracker that records the patient's movement and rest patterns, and the control unit adjusts the timing or nature of prompts on the patient communication device based on detected sleep cycles or wakefulness (such that the patient is not disturbed during deep sleep or is appropriately engaged upon waking).

10. The communication system of claim 7, wherein the control unit correlates the physiological data with the patient's communication interactions to assess the patient's well-being, and automatically prioritizes certain messages or adds supplementary information to the caregiver's notifications. For example, if a high heart rate or abnormal vital sign is detected concurrent with a patient request, the system marks the transmitted message to the caregiver as urgent or critical.

11. The communication system of claim 7, further comprising a remote server or cloud-based analytics module connected to the control unit, wherein the remote server receives the physiological data and patient interaction data and performs advanced analyses or machine learning-based predictions of the patient's health status, and wherein results of these analyses (including any predicted adverse events or suggested adjustments in care) are communicated back to the caregiver communication device or integrated into the system's adaptive adjustments.

12. The communication system of claim 7, wherein the wearable sensor data includes motion or posture information, and the control unit detects if the patient is attempting to move or indicates restlessness. In response, the system either prompts the patient (via the patient communication device) with an inquiry if assistance is needed or directly alerts a caregiver that the patient may require attention (for example, if the patient is trying to get out of bed or is excessively fidgeting, indicating discomfort).

13. A communication method for cross-linguistic messaging between a nonverbal patient and a caregiver, the patient and caregiver having different primary languages, the method comprising:
(a) displaying on a patient communication device a set of symbols or prompts in a first language understood by the patient;
(b) receiving, via a nonverbal input interface of the patient communication device, a selection of one of the symbols by the patient, the selected symbol corresponding to a predetermined message;
(c) determining a second language preferred by the caregiver and translating the predetermined message into the second language;
(d) transmitting the patient's message, translated into the second language, to a caregiver communication device; and
(e) presenting the translated message on the caregiver communication device to notify the caregiver of the patient's need, such that the caregiver perceives the message in the caregiver's language, thereby facilitating cross-linguistic communication.

14. The communication method of claim 13, wherein presenting the translated message on the caregiver communication device comprises displaying text in the second language and/or audibly outputting a spoken version of the message in the second language, so that the caregiver can read or hear the patient's request in a language the caregiver understands.

15. The communication method of claim 13, further comprising receiving a caregiver input on the caregiver communication device (the input being in the second language) as a response or query to the patient, translating the caregiver's input into the first language, and presenting the translated caregiver input on the patient communication device in the first language, thereby enabling two-way communication between the patient and caregiver across the two languages.

16. The communication method of claim 13, wherein the set of symbols on the patient communication device is associated with a library of predetermined phrases stored in multiple languages, and the translating step (c) comprises looking up a corresponding phrase in the caregiver's second language for the selected symbol.

17. The communication method of claim 13, wherein step (d) further comprises identifying the caregiver's device or user profile to determine the appropriate second language, and automatically selecting the translation based on that profile without requiring manual language selection, so that the system dynamically supports multiple caregivers each potentially using a different language.

18. The communication method of claim 13, wherein the translating step utilizes a machine translation algorithm if a selected message or caregiver response does not exactly match a stored phrase, and wherein the method includes verifying the translated content for critical communications to ensure accuracy (optionally by providing the original and translated text to a caregiver for confirmation before presentation to the patient).
